(19) United States
(12) Patent Application Publication
(10) Pub. No.: US 2008/0166396 A1
(43) Pub. Date: Jul. 10, 2008

(54) Boronate Medicaments Suitable for Short Duration Anticoagulation

(75) Inventors: Guy Michael Patrick, Crowthorne (GB); Sophie Marie Combe-Marzelle, Paris (FR); Anthony James Kennedy, London (GB); Roger Withington, Surrey (GB); Oliver Vimpany Arnold Boucher, Brussels (BE)

Correspondence Address: STERNE, KESSLER, GOLDSTEIN & FOX P.L.L.C., 1100 NEW YORK AVENUE, N.W., WASHINGTON, DC, 20005, US

(73) Assignee: Trigen Holdings AG Clareville House, London (GB)

(21) Appl. No.: 10/591,962

(22) PCT Filed: Mar. 9, 2005
(86) PCT No.: PCT/GB05/00907

§ 371 Date: Sep. 19, 2007

(30) Foreign Application Priority Data
Mar. 9, 2004 (GB) 0405272.6

(51) Int. Cl. A61K 9/70; A61K 9/46

(52) U.S. Cl. 424/443; 514/64; 424/489; 424/466; 514/19

(57) ABSTRACT

An oral dosage form of a compound selected from boronic acids which have a neutral thrombin (P1) domain linked to a hydrophobic moiety capable of binding to the thrombin (S2) and (S3) subsites, and salts, prodrugs and prodrug salts of such acids, the dosage form comprising a solid phase formulation comprising the compound and being adapted for reconstitution of the formulation to form a liquid preparation.

### BACKGROUND

The present disclosure relates to substances selected from organoboronic acids and pharmaceutically useful products obtainable therefrom. The disclosure also relates to the use of members of the aforesaid class of substances, to their formulation, their dosage forms and to other subject matter.

### Boropeptide Serine Protease Inhibitors

Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases.

In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem. Biophys. Res. Comm., 27:157-162, 1967. In thrombin, the S1 binding site or “specificity pocket” is a well defined slit in the enzyme, whilst the 52 and S3 binding subsites (also respectively called the proximal and distal hydrophobic pockets) are hydrophobic and interact strongly with, respectively, Pro and (R)-Phe, amongst others.

Aminoboronate or peptidoboronate inhibitors or substrates of serine proteases are described in:
U.S. Pat. No. 4,935,493
EP 341661
WO 94/25049
WO 95/09859
WO 96/12499
WO 96/20689
Lee S-L et al, Biochemistry 36:13180-13186, 1997
Dominguez C et al, Bioorg. Med. Chem. Lett 7:79-84, 1997
EP 471651
WO 94/20526
WO 95/20603
WO97/05161
U.S. Pat. No. 4,450,105
U.S. Pat. No. 5,106,948
U.S. Pat. No. 5,169,841
WO 96/25427
U.S. Pat. No. 5,288,707
WO 96/20698
WO 01/02424.

The amino acid sequence (R)-Phe-Pro-Arg, imitating amino acid sequences of fibrinogen, was at one time considered the best sequence for thrombin inhibitors. This sequence formed tight-binding inhibitors of thrombin, e.g. Ac—(R)-Phe-Pro-boroArg (DUP 714), having Ki values in the picomolar range (Kettner et al, J. Biol. Chem. 265: 18289-18297, 1990; EP-A-293,881).

The replacement of the P2 Pro residue of borotripeptide thrombin inhibitors by an N-substituted glycine is described in Fevig J M et al Bioorg. Med. Chem. 8: 301-306 and Rupin A et al Thromb. Haemost 78(4):1221-1227, 1997. See also U.S. Pat. No. 5,585,360 (de Nanteuil et al).

Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Unfortunately, organoboronic acids can be relatively difficult to obtain in analytically pure form. Thus, alkylboronic acids and their boroxines are often air-sensitive. Korcek et al, J. Chem. Soc. Perkin Trans. 2:242, 1972, teaches that butylboronic acid is readily oxidized by air to generate 1-butanol and boric acid.

It is known that derivatisation of boronic acids as cyclic esters provides oxidation resistance. For example, Martichonok V et al J. Am. Chem. Soc. 118: 950-958, 1996 state that diethanolamine derivatisation provides protection against possible boronic acid oxidation. U.S. Pat. No. 5,681,978 (Matteson D S et al) teaches that 1,2-diols and 1,3 diols, for example pinacol, form stable cyclic boronic esters that are not easily oxidised.

WO 02/059131 discloses boronic acid products which are described as stable. In particular, these products are certain boropeptides and/or boropeptidomimetics in which the boronic acid group has been derivatised with a sugar, e.g. mannitol, to form a sugar ester.

### Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b). The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem J. 290:309-312, 1993
Tapparelli C et al, J Biol Chem, 268:4734-4741, 1993
Claeson G, in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol Sci. 14:366-376, 1993
Claeson G, Blood Coagulation and Fibrinolysis 5:411-436, 1994
Elgendy et al, Tetrahedron 50:3803-3812, 1994
Deadman J et al, J. Enzyme Inhibition 9:29-41, 1995
Deadman J et al, J. Medicinal Chemistry 38:1511-1522, 1995.

TRI 50b is considered to be a prodrug for TRI 50c, which is the active principal in vivo. The tripeptide sequence of TRI 50c has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. Thus, the active, or most active, TRI 50c stereoisomer is considered to be of R,S,R configuration and may be represented as:

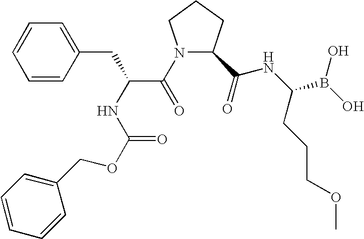

(R,S,R)-TRI 50c Cbz-(R)-Phe-(S)-Pro-(R)-Mpg-B(OH)₂

PCT/GB03/03897, and also U.S. Ser. No. 10/659,178 and EP-A-1396270, disclose pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A),

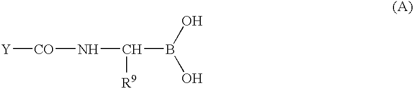

wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —CH₂)ₘ—W where m is 2, 3, 4 or 5 (e.g. 4) and W is OH or halogen (F, Cl, Br or I). R⁹ is an alkoxyalkyl group in one subset of compounds, e.g. alkoxyalkyl containing 4 carbon atoms. Salts of TRI 50c are exemplary.

Also disclosed are oral formulations of such salts.

The salts are described as being of relative stability to hydrolysis and deboronation.

PCT/GB03/03887, and also U.S. Ser. No. 10/659,179 and EP-A-1396269, disclose salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Such drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above. Salts of TRI 50c are exemplary.

PCT/GB03/03883, and also U.S. Ser. No. 10/658,971 and EP-A-1400245, disclose and claim inter alia parenteral pharmaceutical formulations that include a pharmaceutically acceptable base addition salt of a boronic acid of, for example, formula (A) above. Such salts are described as having an improved level of stability which cannot be explained or predicted on the basis of known chemistry. Salts of TRI 50c are exemplary.

### Oral Dosage Forms

Orally administered drugs are often presented as tablets or capsules for swallowing. Other dosage forms are known, however.

Thus an orally administered drug may be presented as reconstitutable formulations, in particular in a form for reconstitution before administration as a liquid and often as drink, for example as an effervescent tablet or in particulate form (as a powder or granules). It is also known for soluble drugs to be packaged as a powder or granules for direct dissolution in the mouth; additionally known are “fast melt” or “fast dissolving” oral formulations, which dissolve or disintegrate rapidly when taken into the mouth. The formulations described in the preceding sentence may also be regarded as reconstitutable formulations in that they are reconstituted in the mouth, prior to the reconstituted formulation reaching the stomach. All these reconstitutable formulations avoid the delays associated with active ingredients in tablets or capsules reaching the blood, as a result of time taken for the tablet/capsule to disintegrate and for its contents to dissolve. Another potential benefit of reconstitutable formulations relates to active ingredients whose required dosage is too high to be incorporated in a single tablet or capsule: the ingestion of multiple tablets or capsules is considered undesirable by patients and might create an additional risk of variation in bioavailability, and the replacement of multiple tablets or capsules with a reconstitutable formulation will avoid these particular shortcomings.

A common form of dosage for the oral administration of drugs is that of particulate formulations contained in dispensing containers, e.g. sachets, particularly monodose sachets. The contents of the dispensing container are usually (but not always) poured out in, for example, a glass of water or in fruit juice or in milk, for drinking by the patient. It is helpful for the particulate formulation to be reconstituted into a drink-size volume (e.g. 50-150 ml, by way of non-limiting example) because this minimises the amount of active which is at risk of being lost to the patient, as compared with a 5 ml volume (in which case the patient will lose a considerable proportion of the active if even a small volume of the liquid is not ingested). Additionally, a larger volume of liquid permits a higher proportion of flavourings to be used, in the event that there are flavour problems.

The dispensing container may in principle be any container which may be opened to release a single dose or a part of a dose. In many instances, a container will be a single dose or monodose container, in which the container contains the correct amount for a single administration of the formulation. Alternatively, the formulation may be presented as a divided dose, in which there is provided a unit dosage which is possibly smaller than some patients require; in this latter case, the patient will take two or more unit doses in a single administration of the formulation. The dispensing container may alternatively be a metered dose container, in which a unit of formulation is metered from a reservoir of the formulation. As an alternative to a sachet, the container may be a plastics container, for example.

Thus, an exemplary unit dosage form for particulate formulations, e.g. for reconstitution as a drink, is the sachet. A sachet is a pouch formed by folding and/or sealing together the edges of a suitable material. As suitable materials may be mentioned thin, flexible sheets of one or more layers that can be sealed together with an adhesive, typically a heat sealable adhesive, or that can be sealed to each other by the application of heat and pressure. Typical materials that can be sealed together by heat and pressure are thermoplastic polymers such as polyethylene and polyvinyl chloride, for example. These polymers may be clear, colourless or coloured and can be made opaque by the addition of suitable opacifying agents such as titanium dioxide. Several layers of materials can be bonded together to form laminates with particular properties. For example a laminate could comprise an outer layer of paper (which can easily be printed on), polyethylene (which provides strength to the laminate), aluminium foil (to act as a barrier that is impermeable to gasses, vapours and liquids) and an inner heat sealable lacquer (to enable the laminate to be sealed to itself or to other materials). Sachets may be formed from a single sheet of laminate or from two sheets that may be the same or different, for example coloured and colourless or clear and opaque.

Sachets may contain solids, powders or liquids that are enclosed by the sachet before the sachet is sealed. In pharmaceutical applications sachets are used to contain powders or granules, typically for reconstitution with water or other liquids.

Particulate formulations suitable for filling into sachets may contain (but are not required to contain) e.g. diluents, flow aids, lubricants, buffering agents, granulating agents, disintegrants, solubilising agents, viscosity enhancers, sweeteners and flavours, in addition to the active ingredient.

Effervescent tablets are another common oral dosage form and contain ingredients that react together in the presence of water to produce carbon dioxide. The liberation of carbon dioxide when effervescent tablets are added to water promotes their disintegration and the dissolution or dispersion of the active ingredients and other components. Effervescent tablets are usually intended to be added to water to produce a solution or dispersion for oral administration. They are usually much bigger than other tablets because they are used for drugs whose dosage is large and they often contain relatively large amounts of flavouring agents.

Ingredients that can react together in water to produce carbon dioxide include organic acids and carbonates. For example citric acid and/or tartaric acid can be combined with calcium carbonate and/or sodium bicarbonate in proportions and quantities that provide the required extent and rate of carbon dioxide production. Other ingredients may be those commonly used in non-effervescent tablet formulations.

The effervescent tablet typically consists of at least three components: the active ingredient; an acid; and an alkali compound (basic ingredient) constituted by a carbonate or a bicarbonate.

In this instance, the acid and the alkali are the essential components which provide the effervescence and the disintegration of the tablet when it is contacted with water. As acidic component citric acid in anhydrous form is often used, but other edible acids like tartaric, fumaric, adipic and malic acid can be used as well. The carbonate, which represents a source of carbon dioxide which generates the effervescence, generally is a water-soluble alkaline carbonate. Sodium bicarbonate is one of the most used carbonates because it is very soluble and of low cost. Alternatively, modified sodium bicarbonate can be used, obtained by heating common sodium bicarbonate in order to convert the surface of its particles to sodium carbonate so increasing its stability.

Other physiologically acceptable alkaline or alkali earth metal carbonates may be used, such as potassium or calcium (bi)carbonate, sodium carbonate, or sodium glycine carbonate.

Compositions of effervescent tablets may also include a tablet lubricant, which may be a water soluble compound forming a clear solution. Examples of this kind of lubricant are sodium benzoate, sodium acetate, polyethylenglycols (PEG) higher than 4000, alanine and glycine.

Conventional excipients such as diluents, ligands, buffers, sweeteners, flavours, colours, solubilizers, disintegrants, wetting agents and other excipients of common use may be added to the formulation. Effervescent tablets are convenient, attractive, easy to use, premeasured dosage forms. These advantages, however, are balanced by hygroscopicity, which usually means that the tablets have to be manufactured in conditions of low relative humidity and packaged in containers that provide good protection against the ingress of water vapour.

Current manufacturers of “fast melt” formulations, i.e. rapidly disintegrating or dissolving solid dose oral formulations, include Cima Labs, Fuisz Technologies Ltd., Prographarm, R. P. Scherer (now part of Cardinal Health), and Yamanouchi-Shaklee. All of these manufacturers market different types of rapidly dissolving solid oral dosage forms.

Cima Labs markets OraSolv®, which is an effervescent direct compression tablet having an oral dissolution time of five to thirty seconds, and DuraSolv®, which is a direct compression tablet having a taste-masked active agent and an oral dissolution time of 15 to 45 seconds. Cima's U.S. Pat. No. 5,607,697, for “Taste Masking Microparticles for Oral Dosage Forms,” describes a solid dosage form consisting of coated microparticles that disintegrate in the mouth. The microparticle core has a pharmaceutical agent and one or more sweet-tasting compounds having a negative heat of solution selected from mannitol, sorbitol, a mixture of an artificial sweetener and menthol, a mixture of sugar and menthol, and methyl salicylate. The microparticle core is coated, at least partially, with a material that retards dissolution in the mouth and masks the taste of the pharmaceutical agent. The microparticles are then compressed to form a tablet. Other excipients can also be added to the tablet formulation.

WO 98/46215 for “Rapidly Dissolving Robust Dosage Form,” assigned to Cima Labs, is directed to a hard, compressed, fast melt formulation having an active ingredient and a matrix of at least a non-direct compression filler and lubricant. A non-direct compression filler is typically not free-flowing, in contrast to a direct compression (DC grade) filler, and usually requires additional processing to form free-flowing granules.

Cima also has U.S. patents and international patent applications directed to effervescent dosage forms (U.S. Pat. Nos. 5,503,846, 5,223,264, and 5,178,878) and tableting aids for rapidly dissolving dosage forms (U.S. Pat. Nos. 5,401,513 and 5,219,574), and rapidly dissolving dosage forms for water soluble drugs (WO 98/14179 for “Taste-Masked Microcapsule Composition and Methods of Manufacture”).

Fuisz Technologies markets Flash Dose®, which is a direct compression tablet containing a processed excipient called Shearform®. Shearform® is a cotton candy-like substance of mixed polysaccharides converted to amorphous fibers. U.S. patents describing this technology include U.S. Pat. No. 5,871,781 for “Apparatus for Making Rapidly Dissolving Dosage Units;” U.S. Pat. No. 5,869,098 for “Fast-Dissolving Comestible Units Formed Under High-Speed/High-Pressure Conditions;” U.S. Pat. Nos. 5,866,163, 5,851,553, and 5,622,719, all for “Process and Apparatus for Making Rapidly Dissolving Dosage Units and Product Therefrom;” U.S. Pat. No. 5,567,439 for “Delivery of Controlled-Release Systems;” and U.S. Pat. No. 5,587,172 for “Process for Forming Quickly Dispersing Comestible Unit and Product Therefrom.”

Prographarm markets Flashtab®, which is a fast melt tablet having a disintegrating agent such as carboxymethyl cellulose, a swelling agent such as a modified starch, and a taste-masked active agent. The tablets have an oral disintegration time of under one minute (U.S. Pat. No. 5,464,632).

R. P. Scherer markets Zydis®, which is a freeze-dried tablet having an oral dissolution time of 2 to 5 seconds. Lyophilized tablets are costly to manufacture and difficult to package because of the tablets sensitivity to moisture and temperature. U.S. Pat. No. 4,642,903 (R. P. Scherer Corp.) refers to a fast melt dosage formulation prepared by dispersing a gas throughout a solution or suspension to be freeze-dried. U.S. Pat. No. 5,188,825 (R. P. Scherer Corp.) refers to freeze-dried dosage forms prepared by bonding or complexing a water-soluble active agent to or with an ion exchange resin to form a substantially water insoluble complex, which is then mixed with an appropriate carrier and freeze dried. U.S. Pat. No. 5,631,023 (R. P. Scherer Corp.) refers to freeze-dried drug dosage forms made by adding xanthan gum to a suspension of gelatin and active agent. U.S. Pat. No. 5,827,541 (R. P. Scherer Corp.) discloses a process for preparing solid pharmaceutical dosage forms of hydrophobic substances. The process involves freeze-drying a dispersion containing a hydrophobic active ingredient and a surfactant, in a non-aqueous phase; and a carrier material, in an aqueous phase.

Yamanouchi-Shaklee markets Wowtab®, which is a tablet having a combination of a low moldability and a high moldability saccharide. U.S. patents covering this technology include U.S. Pat. No. 5,576,014 for “Intrabuccally Dissolving Compressed Moldings and Production Process Thereof,” and U.S. Pat. No. 5,446,464 for “Intrabuccally Disintegrating Preparation and Production Thereof.”

Other companies owning rapidly dissolving technology include Janssen Pharmaceutica. U.S. patents assigned to Janssen describe rapidly dissolving tablets having two polypeptide (or gelatin) components and a bulking agent, wherein the two components have a net charge of the same sign, and the first component is more soluble in aqueous solution than the second component. See U.S. Pat. No. 5,807,576 for “Rapidly Dissolving Tablet;” U.S. Pat. No. 5,635,210 for “Method of Making a Rapidly Dissolving Tablet;” U.S. Pat. No. 5,595,761 for “Particulate Support Matrix for Making a Rapidly Dissolving Tablet;” U.S. Pat. No. 5,587,180 for “Process for Making a Particulate Support Matrix for Making a Rapidly Dissolving Tablet;” and U.S. Pat. No. 5,776,491 for “Rapidly Dissolving Dosage Form.”

Eurand America, Inc. has U.S. patents directed to a rapidly dissolving effervescent composition having a mixture of sodium bicarbonate, citric acid, and ethylcellulose (U.S. Pat. Nos. 5,639,475 and 5,709,886).

L.A.B. Pharmaceutical Research owns U.S. patents directed to effervescent-based rapidly dissolving formulations having an effervescent couple of an effervescent acid and an effervescent base (U.S. Pat. Nos. 5,807,578 and 5,807,577).

Schering Corporation has technology relating to buccal tablets having an active agent, an excipient (which can be a surfactant) or at least one of sucrose, lactose, or sorbitol, and either magnesium stearate or sodium dodecyl sulfate (U.S. Pat. Nos. 5,112,616 and 5,073,374).

Takeda Chemicals Inc., Ltd. owns technology directed to a method of making a fast dissolving tablet in which an active agent and a moistened, soluble carbohydrate are compression molded into a tablet, followed by drying of the tablets.

In practice, oral and intravenous presentations of active ingredients are completely distinct. In particular, it is desirable in the case of an intravenous formulation that any unnecessary excipients and components additional to the active ingredient be kept to a minimum. In contrast, oral formulations contain additional excipients. The class and identity of excipient will vary with the type of oral formulation but they may contain any one or more of the following:
antimicrobial preservatives, flow aids (glidants), surfactants, flavour agents (in the case of powders)
antimicrobial preservatives, flow aids (glidants), surfactants, viscosity enhancers, binders, disintegrants, flavour agents (in the case of granules)
antimicrobial preservatives, tablet lubricants, surfactants, viscosity enhancers, binders, disintegrants, swelling agents (in the case of tablets)
antimicrobial preservatives, tablet lubricants, surfactants, binders, swelling agents, disintegrating agents, flavour agents (“fast melt” formulations).

The reader is referred to the US Pharmacopeia for more detailed information as to members of the above classes of excipient.

Another characteristic of oral formulations is that they are not required to be, and usually are not, isotonic. In contrast, intravenous formulations are isotonic and in most cases have pH of between 4 and 5.

### Rapid Onset Anticoagulation and Short Duration Anticoagulation

It is desirable in some circumstances to administer an anticoagulant which will have a relatively short duration of significant activity (have rapid offset). Thus, some anticoagulants will have an excessively long duration of activity for a particular indication; one example is that warfarin takes a long time to reduce from therapeutic levels of activity and is unsuitable to haemodialysis, where a short duration anticoagulant is desirable.

Haemodialysis is used to treat patients in end stage renal failure. An anticoagulant is administered to the patient prior to commencement of dialysis, in order to prevent thrombosis in the haemodialysis circuit. Anticoagulation is required only for the duration of the haemodialysis session (typically about four hours) and the anticoagulant should desirably have little or no effect after the patient has left the clinic. For maximum convenience, the anticoagulant should not be one which is renally cleared because anticoagulation lasts longer when the kidney is impaired either requiring patient-specific dose adjustment of the anticoagulant and/or increased monitoring of coagulation status during the session and/or retention of the patient within the clinic for longer than the session to allow coagulation parameters to return to safe levels. Such haemodialysis (“HD”) in which the patient has periodic haemodialysis sessions of limited duration (e.g. four hours) may be described as “chronic intermittent” haemodialysis (CIHD), in distinction to acute HD which can be continuous for extended periods in patients who are very ill e.g. with acute kidney failure or other major injury and on life support; obviously oral anticoagulant is not a convenient option for acute patients. CIHD is an example of intermittent apheresis, in which a patient is intermittently subjected to apheresis of limited duration, as opposed to continuous apheresis. Apheresis is a process which involves removal of whole blood from a subject (it may be a patient or a donor), components of the blood are separated, one of the separated portions is then withdrawn and the remaining components are retransfused into the subject; the withdrawn components may be toxins.

Currently, anticoagulation in haemodialysis is primarily achieved by injections and/or infusion of heparins (including low molecular weight heparin). Heparin has a variable response and can easily be over or underdosed resulting in bleeding, which is potentially dangerous, or in insufficient anticoagulation which causes clotting in the filter (“artificial kidney”) so reducing the efficiency of the session. Anticoagulation monitoring of the patient is therefore desirable. When bleeding occurs, it must be treated and when a filter clots it must be flushed or changed. In home haemodialysis, the complexity and responsibility of self-management or partner/carer-management of intravenous anticoagulation is an additional challenge among the many faced by patients contemplating this option. The availability of an oral anticoagulant would reduce this element of complexity.

Other occasions where short term oral anticoagulation would be desirable include flight DVT (deep vein thrombosis), in the case of aeroplane passengers believed to be at risk of DVT, and in apheresis or elective extracorporeal blood detoxification procedures. An exemplary extracorporeal blood detoxification procedure is liver detoxification, i.e. extracorporeal blood detoxification in the case of liver failure. Other disorders in which therapeutic apheresis is performed include autoimmune diseases (to remove antibodies); in these cases, patients may have plasmapheresis, in which removal of plasma (and its replacement by saline solution) will help to reduce circulating antibodies and immune complexes. Apheresis may additionally be used to remove low density lipoprotein, in patients at suffering from or at risk of cardiovascular disease, platelets or leukocytes. Also to be mentioned is donation by apheresis.

Flight DVT is a rare condition. Nevertheless, for people not requiring oral anticoagulation in the normal course of events but who, during a long haul flight might become susceptible to DVT, it would be desirable to have a rapid onset anticoagulant agent available orally for patients to take at the beginning of a flight or shortly after take-off. For longer flights, a second dose can be taken at a fixed time after the first in order to ensure coverage throughout the flight.

For certain elective blood cleansing or apheresis procedures, where the patient knows in advance that he or she is due to have blood passed through an extracorporeal circuit, it may be preferable and more convenient for the patient to receive his or her own anticoagulant orally to cover the duration of the procedure and to avoid the complexity and management of the solution anticoagulation normally required in such procedures.

### BRIEF SUMMARY OF THE DISCLOSURE

The present disclosure relates in one aspect to novel oral formulations which are capable of providing a relatively short duration of activity. It also relates to novel indications for treatment by certain boronic acids and derivatives thereof.

The disclosure also relates to novel organoboronic acids and their derivatives, including those described in more detail later in this specification under the heading “Novel Boronic Acids”.

The disclosure additionally concerns compounds selected from organoboronic acid thrombin inhibitors, their salts, prodrugs and prodrug salts, and in one aspect relates to novel oral dosage forms of such thrombin inhibitors. The disclosure further concerns dosage forms of such thrombin inhibitors which dosage forms are useful for administration when relatively rapid onset and/or short duration of activity is required, e.g. anticoagulant therapy during haemodialysis. The thrombin inhibitors may comprise pharmaceutically acceptable base addition salts of boronic acids as described in more detail below. The boronic acids include those having a neutral thrombin P1 domain linked to a hydrophobic moiety capable of binding to the thrombin S2 and S3 subsites. Particular thrombin inhibitors are pharmaceutically acceptable base addition salts of such boronic acids, e.g. those which have a neutral aminoboronic acid residue capable of binding to the thrombin S1 subsite linked to a hydrophobic moiety capable of binding to the thrombin S2 and 53 subsites, e.g. those of formula (I) below.

The disclosure provides oral dosage forms of the boronic acid compounds (e.g. salts) described herein in a solid phase formulation, the dosage form being adapted for entry of the active ingredient into a liquid vehicle prior to entering the stomach. Thus, the solid phase formulation may be adapted for reconstitution into a solution or, less preferably, a suspension prior to administration; alternatively it may be a “fast melt” formulation which disintegrates or dissolves rapidly in the mouth. For a general description of the characteristics of the formulation types to which the disclosure relates, the reader is referred to the earlier section of this specification under the heading “Oral Dosage Forms”.

Many solid phase formulations of the disclosure contain an anti-microbial preservative. Commonly, solid phase formulations of the disclosure contain a flavour agent (e.g. flavouring, flavour enhancer or sweetener). Powders often contain a flow aid or glidant. Granules often contain a binder. In general but not invariably, the solid phase formulations of the disclosure will contain both an anti-microbial preservative and a flavour agent, usually in uniform distribution; this of course is different from a parenteral formulation. Powders and granules may contain an organic acid; effervescent tablets in practice do so.

The dosage form may for example be a sachet or other sealed dispensing container containing a particulate formulation (powder or granules) or it may be an effervescent tablet or a “fast melt” formulation.

Also provided is the use of a boronic acid compound disclosed herein (e.g. salt) for the manufacture of a solid phase medicament for combining with a suitable liquid to form a liquid preparation for oral ingestion for use in treating thrombosis. The disclosure includes an embodiment in which the liquid preparation is a drinkable preparation. A liquid suitable to combine with the solid phase medicament is non-toxic and does not seriously detract from the activity or bioavailability of the drug; in practice, the taste of the liquid must not be excessively unpalatable.

One class of methods of the disclosure comprises a method of preparing an anticoagulant preparation, comprising reconstituting, into a liquid preparation for oral administration and preferably a drinkable preparation, a solid phase formulation comprising a boronic acid compound disclosed herein, e.g. a pharmaceutically acceptable base addition salt of a boronic acid which has a neutral thrombin P1 domain linked to a hydrophobic moiety capable of binding to the thrombin S2 and S3 subsites.

Included in the disclosure is a method of inhibiting thrombin in the treatment of disease, comprising administering perorally to a subject in need thereof a therapeutically effective amount of a boronic acid compound disclosed herein, e.g. pharmaceutically acceptable base addition salt of a boronic acid which has a neutral thrombin P1 domain linked to a hydrophobic moiety capable of binding to the thrombin 52 and 53 subsites, said compound being put into solution or suspension from a solid phase formulation prior to the salt entering the stomach. In one class of methods, the compound is put into solution or suspension by reconstitution with a liquid prior to administration; in another class of methods the compound is put into solution or suspension in the mouth.

Additionally included are methods of preventing one of:
thrombosis in the haemodialysis circuit of patients having haemodialysis
flight DVT
thrombosis in apheresis, e.g. extracorporeal liver detoxification (see above).

The examples of this specification indicate that the disclosed compounds are, surprisingly, absorbed relatively rapidly from the stomach without damaging levels of degradation of the active principle and that plasma concentrations of the active principle return to tolerably low levels within about 4 hours of administration. In an experiment involving the monosodium salt of TRI 50c, it was found that only 14% of the administered dose was excreted as TRI 50c species. It will be appreciated that such short duration of action (rapid offset of activity) of activity is well suited for the prevention of thrombosis in intermittent apheresis procedures, and that oral administration is particularly useful in the case of elective intermittent apheresis procedures. In one class of embodiments, the apheresis procedure is haemodialysis (strictly CIHD); in another, the boronic acid is administered in the form of a base addition salt and the procedure is not haemodialysis.

The compounds are described in more detail below with particular reference to base addition salts. However, the methods are not limited to the disclosed salts but may use any boronic acid disclosed herein, or any salt (e.g. acid addition salt), prodrug or prodrug salt thereof. The methods comprise administering to a subject in need thereof a therapeutically effective amount of a compound selected from boronic acids which have a neutral thrombin P1 domain linked to a hydrophobic moiety capable of binding to the thrombin S2 and S3 subsites and salts, prodrugs and prodrug salts thereof. In these methods, the disclosure is not limited as to the route of administration or the nature of the formulation; for example, in these methods the compound may be administered parenterally, e.g. intravenously or by the more preferred oral route; where oral administration is chosen, the active compound may be administered in the form of a tablet or capsule, for example, but it may otherwise be administered as a reconstitutable formulation. In one class of these methods, the selected compound is not a base addition salt; in another class it is a base addition salt.

In other aspects, the disclosure includes the use of a boronic acid compound described herein for the manufacture of a medicament for preventing thrombosis in the haemodialysis circuit of patients having haemodialysis, flight DVT or thrombosis in extracorporeal liver detoxification (per above). The medicament may be adapted for oral administration, e.g. a tablet, capsule or reconstitutable formulation; alternatively, it may be adapted for parenteral administration.

The disclosure includes products comprising a solid phase formulation containing a boronic acid compound described herein, the product being adapted for reconstitution of the formulation into a drinkable preparation to prevent thrombosis during haemodialysis.

The disclosure includes products comprising a solid phase formulation containing a boronic acid compound described herein, the product being adapted for reconstitution of the formulation into a drinkable preparation for emergency treatment of actual or suspected thrombosis e.g. prior to proper medical examination of the patient.

The solid phase formulations may include a boronic acid of formula (I) below or a salt or prodrug thereof, or a salt of a prodrug. As prodrugs may be mentioned esters, e.g. with a residue of an alkanol, e.g. a C₁-C₄ alkanol such as methanol or ethanol, for example. Also to be mentioned are cyclic derivatives, in which the two available valencies of the boron (corresponding to the bonds to the two —OH groups of the free acid) are bonded to respective ends of a chain of atoms, i.e. the boron becomes part of a ring. Such cyclic derivatives may be represented as below in the case of acids of Formula (I), modified mutatis mutandis for acids of other formulae disclosed herein:

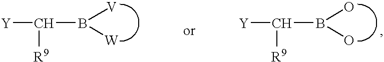

where V and W are heteroatoms (e.g. selected independently from N, O and S) and the arcuate line represents a linear or branched chain of atoms, the length of the chain between the two bonds from the boron is not critical but may be 4, 5 or 6 in some cases. As described, the chain terminated at both ends by the boron (the ring-forming chain) may be linear or branched, e.g., it may have one or more side branches; where there are multiple side branches, at least some of them may join together to form a ring, as in the case of pinanediol esters, for example.

Particular cyclic derivatives, therefore, are cyclic esters formed by diols. The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexyl-ethanediol.

The prodrug may be a sugar derivative as described in WO 02/059131 and equivalent U.S. Pat. No. 6,699,835 (see above). Thus, the boronate group may be esterified with a sugar such as a monosaccharide or a disaccharide, for example. The sugar may be a reduced sugar, e.g. manittol or sorbitol: it may be an individual sugar or class of sugars taught in WO 02/059131. The boronic acid, sugar (or other diol) and water may be combined and then lyophilised, for example as taught in WO 02/059131.

Salts may be acid addition salts or base addition salts.

A particular class of soluble compounds comprises alkali metal salts, sugar esters and amino sugar salts of the disclosed boronic acids. Such soluble derivative are useful for making formulations for reconstitution as solutions. Another particular class comprises the free acids. Also to be mentioned are acid addition salts.

The boronic acids with which the disclosure is concerned are thrombin inhibitors and are, for example, of formula (I), and their salts, prodrugs and prodrug salts. There is disclosed also a solid phase pharmaceutical formulation for use in preparing a liquid oral preparation, preferably a drinkable preparation, therefrom that includes a boronic acid of, for example, formula (I) or a salt, prodrug or prodrug salt thereof:

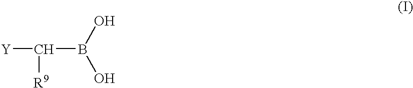

wherein
Y comprises a moiety which, together with the fragment —CH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

In some embodiments, Y comprises
an amino group bonded to structural fragment —CH(R⁹)—B(OH)₂, and
a hydrophobic moiety which is linked to said amino group and which, together with said structural fragment, has affinity for the substrate binding site of thrombin.

The disclosure includes products comprising active ingredients which are base addition salts of hydrophobic boronic acid inhibitors of thrombin. Such inhibitors may contain hydrophobic amino acids, and this class of amino acids includes those whose side chain is hydrocarbyl, hydrocarbyl containing an in-chain oxygen and/or linked to the remainder of the molecule by an in-chain oxygen or heteroaryl, or any of the aforesaid groups when substituted by hydroxy, halogen or trifluoromethyl. Representative hydrophobic side chains include alkyl, alkoxyalkyl, either of the aforesaid when substituted by at least one aryl or heteroaryl, aryl, heteroaryl, aryl substituted by at least one alkyl and heteroaryl substituted by at least one alkyl. Proline and other imino acids which are ring-substituted by nothing or by one of the moieties listed in the previous sentence are also hydrophobic.

Some hydrophobic side chains contain from 1 to 20 carbon atoms, e.g. non-cyclic moieties having 1, 2, 3 or 4 carbon atoms. Side chains comprising a cyclic group typically but not necessarily contain from 5 to 13 ring members and in many cases are phenyl or alkyl substituted by one or two phenyls.

Included are inhibitors which contain hydrophobic non-peptide moieties, which are typically based on moieties which may form a side chain of a hydrophobic amino acid, as described above.

Hydrophobic compounds may contain, for example, one amino group and/or one acid group (e.g. —COOH, —B(OH)₂). Generally, they do not contain multiple polar groups of any one type.

The disclosure comprises products, methods and uses involving or including hydrophobic boronic acid inhibitors of thrombin or their salts or prodrugs, and therefore includes peptide boronic acids which have a partition coefficient between 1-n-octanol and water expressed as log P of greater than 1.0 at physiological pH and 25° C., and the salts and prodrugs thereof. Some useful peptide boronic acids have a partition coefficient of at least 1.5. A class of useful hydrophobic peptide boronic acids has a partition coefficient of no more than 5.

Some sub-classes of hydrophobic organoboronic acids are those described by Formulae (II) and (III) below, under the heading “Detailed Description of Several Examples”.

There is a debate in the literature as to whether boronates in aqueous solution form the ‘trigonal’ B(OH)₂ or ‘tetrahedral’ B(OH)₃— boron species, but NMR evidence seems to indicate that at a pH below the first pKa of the boronic acid the main boron species is the neutral B(OH)₂. In the duodenum the pH is likely to be between 6 and 7, so the trigonal species is likely to be predominant here. In any event, the symbol —B(OH)₂ includes tetrahedral as well as trigonal boron species, and throughout this specification symbols indicating trigonal boron species embrace also tetrahedral species. The symbol may further include boronic groups in anhydride form.

The compounds, e.g. salts, may be in the form of solvates, particularly hydrates.

The base addition salts may comprise, or consist essentially of, acid salts in which the boronic acid is singly deprotonated. The disclosure therefore includes products having a metal/boronate stoichiometry consistent with the boronate groups in the product predominantly (more than 50 mol %) carrying a single negative charge.

The salts may have a purity, e.g. as determined by the method of Example 34, of at least about 90%, e.g. of greater than or equal to about 95%. In the case of pharmaceutical formulations, such salt forms may be combined with pharmaceutically acceptable diluents, excipients or carriers.

According to a further aspect of the present disclosure, there is provided a method of treatment of a condition where anti-thrombotic activity is required which method comprises oral administration by drinking of a therapeutically effective amount of a boronic acid compound disclosed herein, e.g. a pharmaceutically acceptable base addition salt of a boronic acid of formula (I) to a person suffering from, or at risk of suffering from, such a condition.

TRI 50c base addition salts are obtained via TRI 50c esters. However, published synthetic routes to TRI 50c esters and thus to TRI 50c give rise to one or more impurities. The methods described below under the heading ‘“High Purity” Synthesis’ (unpublished as of the priority date of this application) for making the salts give rise to one or more impurities and very high purity salts were not obtained. Further, the salts have proved most challenging to obtain in high purity. Thus, purification techniques which were applied failed to produce very high purity salts. HPLC will not be usable on an industrial scale to purify salts made via published TRI 50c ester syntheses and the salt preparation techniques described under the heading ‘“High Purity” Synthesis’. In other words, in order for the therapeutic benefits of TRI 50c salts to be provided to those in need thereof, the salts must be obtainable industrially in adequately pure form and the pure form must be attainable without the use of excessively expensive purification techniques.

Further aspects and embodiments of the disclosure are described and claimed in the following specification.

The salts described herein include products obtainable by (having the characteristics of a product obtained by) reaction of the boronic acid with a strong base and the term “salt” herein is to be understood accordingly. The term “salt” in relation to the disclosed products, therefore, does not necessarily imply that the products contain discrete cations and anions and is to be understood as embracing products which are obtainable using a reaction of a boronic acid and a base. The disclosure embraces products which, to a greater or lesser extent, are in the form of a coordination compound. The disclosure thus provides also products obtainable by (having the characteristics of a product obtained by) reaction of a disclosed boronic acid with a strong base a well as the therapeutic, including prophylactic, use of such products.

The present disclosure is not limited as to the method of preparation of the salts, provided that they contain a boronate species derived from a disclosed boronic acid and a counter-ion. Such boronate species may be boronate anions in any equilibrium form thereof. The term “equilibrium form” refers to differing forms of the same compounds which may be represented in an equilibrium equation (e.g. boronic acid in equilibrium with a boronic anhydride and in equilibrium with different boronate ions). Boronates in the solid phase may form anhydrides and the disclosed boronate salts when in the solid phase may comprise boronate anhydrides, as a boronic equilibrium species. It is not required that the salts be prepared by reaction of a base containing the counter-ion and the boronic acid. Further, the disclosure includes salt products which might be regarded as indirectly prepared by such an acid/base reaction as well as salts obtainable by (having the characteristics of products obtained by) such indirect preparation. As examples of possibly indirect preparation may be mentioned processes in which, after initial recovery of the salt, it is purified and/or treated to modify its physicochemical properties, for example to modify solid form or hydrate form, or both.

In some embodiments, the cations of the salts are monovalent.

In some embodiments the salts comprise anhydride species; in others they are essentially free of anhydride species.

Further aspects and embodiments of the disclosure are set forth in the following description and claims. Also included as such are the salts described herein.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

This patent application contains data indicating that the stability (resistance to deboronation) of organoboronic acids may be increased by providing them in the form of salts, e.g. metal salts. In single experiments, the ammonium salt of TRI 50c appeared to decompose on drying to yield ammonia, whilst the choline salt demonstrated rapid decomposition to a deboronated impurity. Although experiments have not been conducted to reproduce these unrepeated observations, there is provided a sub-class in which the ammonium and choline salts are excluded. The salt may be an acid salt. In any event, this stabilisation technique forms part of the disclosure and is applicable, inter alla, to organoboronic acids described under the heading “BACKGROUND” and to organoboronic acids described in publications mentioned under that heading.

### DETAILED DESCRIPTION OF SEVERAL EXAMPLES

### Glossary

The following terms and abbreviations are used in this specification:

The expression “acid salt” as applied to a salt of a boronic acid refers to salts of which a single —OH group of the trigonally-represented acid group —B(OH)₂ is deprotonated. Thus salts wherein the boronate group carries a single negative charge and may be represented as —B(OH)(O⁻) or as [—B(OH)₃]⁻ are acid salts. The expression encompasses salts of a cation having a valency n wherein the molar ratio of boronic acid to cation is approximately n to 1. In practical terms, the observed stoichiometry is unlikely to be exactly n:1 but will be consistent with a notional n:1 stoichiometry. For example, the observed mass of the cation might vary from the calculated mass for a n:1 stoichiometry by no more than about 10%, e.g. no more than about 7.5%; in some cases an observed mass of a cation might vary from the calculated mass by no more than about 1%. Calculated masses are suitably based on the trigonal form of the boronate. (At an atomic level, a salt stoichiometrically consistent with being an acid salt might contain boronates in a mix of protonation states, whose average approximates to single deprotonation and such “mixed” salts are included in the term “acid salt”). Examples of acid salts are monosodium salts and hemicalcium salts.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The expression “dispensing container” refers to a container adapted to dispense a contained formulation prior to administration. Thus a capsule, which is not intended to release the active until after ingestion, is not a dispensing container. As used herein, the expression refers particularly to containers which serve to dispense particulate formulations which are to be reconstituted into a liquid formulation or directly placed in the mouth for fast release of the active ingredient. A dispensing container may comprise a moulded plastics body (whether it contains a single unit of formulation or is a metered dose dispenser) or it may be a sachet, for example.

“Drinkable preparation” means a preparation having suitable characteristics to be drunk. Thus the quantity is suitable for drinking (as opposed to a 5 ml spoonful which is swallowed, not drunk) and may by way of example be from about 50 to about 150 ml; sometimes the quantity exceeds 150 ml. The quality must also be suitable for drinking, thus the preparation must be non-toxic and tolerable to drink in terms of taste.

The expression “monodose formulation” means a formulation containing an active ingredient in an amount corresponding to the desired dosage at which the ingredient is to be administered. For example, if at any one instance of administration (e.g. prior to haemodialysis) an active ingredient is to be administered in an amount of 300 mg then a monodose formulation will contain 300 mg of the ingredient.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=His=histidine
K=Lys=lysine

Negatively Charged Amino Acids
D=Asp=aspartic acid
E=Glu=glutamic acid.
   ACN=acetonitrile
   Amino acid=amino acid
   Acid addition salt=a salt which is prepared from addition of an inorganic acid or an organic acid to a free base (e.g. an amino group, as for example an N-terminal amino group of a peptide).
   Base addition salt=a salt which is prepared from addition of an inorganic base or an organic base to a free acid (in this case the boronic acid).
   Cbz=benzyloxycarbonyl
   Cha=cyclohexylalanine (a hydrophobic unnatural amino acid)
   Charged (as applied to drugs or fragments of drug molecules, e.g. amino acid residues)=carrying a charge at physiological pH, as in the case of an amino, amidino or carboxy group
   Dcha=dicyclohexylalanine (a hydrophobic unnatural amino acid)
   Dpa=diphenylalanine (a hydrophobic unnatural amino acid)
   Drug=a pharmaceutically useful substance, whether the active in vivo principle or a prodrug
   Mpg=3-methoxypropylglycine (a hydrophobic unnatural amino acid)
   Multivalent=valency of at least two, for example two or three
   Neutral (as applied to drugs or fragments of drug molecules, e.g. amino acid residues)=uncharged=not carrying a charge at physiological pH
   Pinac=Pinacol=2,3-dimethyl-2,3-butanediol
   Pinanediol=2,3-pinanediol=2,6,6-trimethylbicyclo[3.1.1]heptane-2,3-diol
   Pip=pipecolinic acid
   Room temperature=25° C.±2° C.
   Strong base=a base having a sufficiently high pKb to react with a boronic acid. Suitably such bases have a pKb of 7 or more, e.g. 7.5 or more, for example about 8 or more
   THF=tetrahydrofuran
   Thr=thrombin

### Conversion Factors

Unless otherwise stated the following conversion factors are used in this specification to convert between moles and mass in grams and for other similar calculations:
the molecular weight of TRI 50c is determined in relation to the trigonal form (molecular weight 525.4)
the molecular weight of TRI 50c monosodium salt is calculated on the basis of the monohydrate (C₂₇H₃₅BN₃O₇)Na H₂O (molecular weight 565.39).

### The Compounds

The disclosure relates to boronic acids which have a neutral aminoboronic acid residue capable of binding to the thrombin SI subsite linked to a hydrophobic moiety capable of binding to the thrombin S2 and S3 subsites. It relates also to prodrugs (e.g. esters) and salts of such acids, particularly base addition salts. The disclosure includes acids of formula (I) above and also those of a sub-class represented by the following formula (II):

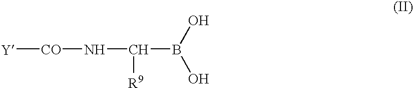

wherein
Y′ comprises a hydrophobic moiety and Y′CO— together with fragment —NHCR(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 (e.g. 5) or R⁹ is —(CH₂)ₘ—W where m is from 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

Reverting now to formula (I), typically, Y— comprises an amino acid residue (whether natural or unnatural) which binds to the S2 subsite of thrombin, the amino acid residue being N-terminally linked to a moiety which binds the 53 subsite of thrombin. Y— may be of the formula Z³-Z²-CO—where -Z²-CO— is an amino acid residue having affinity for the S2 subsite of thrombin and Z³ is a moiety which has affinity for the 53 subsite of thrombin.

The boronic acid may comprise linkages between the structural fragment —CH(R⁹)—B(OH)₂ and moiety Y or linkages and/or a linkage within Y, e.g. the Z³-Z² linkage, which comprises a nitrogen atom as —NH— or as —NR¹⁴— where R¹⁴ is a C₁-C₁₃ hydrocarbyl group optionally containing in-chain and/or in-ring nitrogen, oxygen or sulfur and optionally substituted by a substituent selected from halo, e.g. F, or a functional group, for example hydroxy. The hydrocarbyl group may contain from 1 to 4 carbon atoms; it may be alkyl or otherwise comprise a moiety bonded to said nitrogen atom which is selected from —CH₂— and halogenated variants thereof, especially fluorinated variants for example —CF₂—.

In one class of Formula (I) acids, Y— is an optionally N-terminally protected dipeptide residue which binds to the 53 and 52 binding sites of thrombin and the peptide linkages in the acid are optionally and independently N-substituted by an R¹⁴ group. The N-terminal protecting group, when present, may be a group X as defined above (other than hydrogen). In many instances, the acid contains no N-substituted peptide linkages; where there is an N-substituted peptide linkage, the substituent is often 1 C to 6 C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where Y— is a dipeptide residue (whether or not N-terminally protected), the 53-binding amino acid residue may be of (R)-configuration and/or the S2-binding residue may of (S)-configuration. The fragment —NHCH(R⁹)—B(OH) may be of (R)-configuration. The disclosure is not restricted to chiral centres of these conformations, however.

In one class of compounds, the side chain of the P3 (S3-binding) amino acid and/or the P2 (S2-binding) amino acid is a moiety other than hydrogen selected from a group of formula A or B:

—(CO)ₐ—(CH₂)_{b}-D_{C}-(CH₂)_{d}-E (A)

—(CO)ₐ—(CH₂)_{b}-D_{C}-Cₑ(E¹)(E²)(E³) (B)

wherein
a is 0 or 1;
e is 1;
b and d are independently 0 or an integer such that (b+d) is from 0 to 4 or, as the case may be,
(b+e) is from 1 to 4;
c is 0 or 1;

### D is O or S;

E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1); in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of embodiments, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of embodiments, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

The disclosure includes salts in which the P3 and/or P2 side chain comprises a cyclic group in which 1 or 2 hydrogens have been replaced by halogen, e.g. F or Cl.

The disclosure includes a class of salts in which the side chains of formula (A) or (B) are of the following formulae (i), (ii) or (iii), or be variants thereof in which one or both phenyl rings of (ii) or the phenyl ring of (iii) are replaced by cyclohexyl or cyclohexenyl:

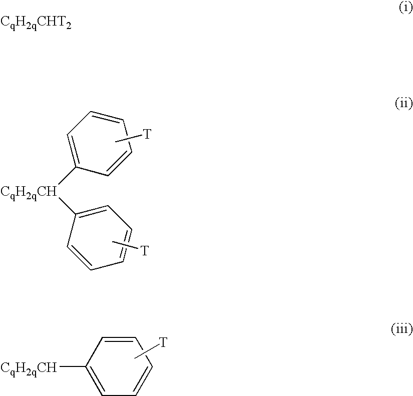

wherein q is from 0 to 5, e.g. is 0, 1 or 2, and each T is independently hydrogen, 1, 2 or 3 halogens (e.g. F or Cl), —SiMe₃, —CN, —R¹³, —OR¹³, —COR¹³, —CO₂R¹³ or —O₂CR¹³. In some embodiments of structures (ii) and (iii), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (I) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many Y— groups which are dipeptide fragments (which dipeptides may be N-terminally protected or not), the P3 amino acid has a side chain of formula (A) or (B) as described above and the P2 residue is of an imino acid.

The disclosure therefore includes medicaments comprising salts, e.g. metal salts, of organoboronic acids which are thrombin inhibitors, particularly selective thrombin inhibitors, having a neutral P1 (S1-binding) moiety. For more information about moieties which bind to the S3, 52 and S1 sites of thrombin, see for example Tapparelli C et al, Trends Pharmacol. Sci. 14: 366-376, 1993; Sanderson P et al, Current Medcinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory salts of the disclosure are not limited to those having S3, S2 and S1 affinity groups described in the publications listed in the preceding sentence. Alternatively to being presented as a salt, the organoboronic acids may be presented as the free acid or a prodrug (e.g. ester).

The boronic acids may have a Ki for thrombin of about 100 nM or less, e.g. about 20 nM or less.

A subset of the Formula (I) acids comprises the acids of Formula (III):

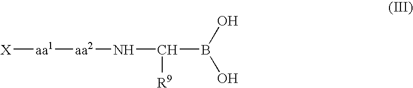

X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0, 1 and 2 are preferred) and R⁶ is H or a 5 to 13-membered cyclic group optionally substituted by one or more (e.g. 1, 2, 3, 4 or 5) halogens (e.g. F), for example at least at the 4-position, and/or by 1, 2 or 3 substituents selected from amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0, 1 or 2 in these moieties, R⁶ may be phenyl or fluorophenyl. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and particularly benzyloxycarbonyl or benzylmethylcarbonyl.

aa¹ is an amino acid residue having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid residue having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

As another alternative, aa² is the β-amino acid analogue of Gly (i.e. H₂N—CH₂—CH₂—COOH) N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

The disclosure includes a class of compounds in which aa² is a residue of a α-amino acid having a 4 to 6 membered carbocyclic ring which optionally has one carbon atom replaced by a sulfur and of which the ring-forming carbon atoms include the carbon atoms α- and β- to the carboxyl group (i.e. the β-amino acid comprises a 4 to 6 membered carbocyclic ring which is 1-substituted by caroboxyl and 2-substituted by amino and which may at one other position contain an S atom.

An exemplary class of products comprises those in which aa² is a residue of an imino acid of formula (IV)

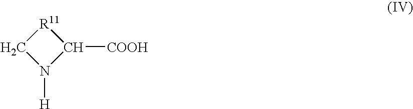

where R¹¹ is —CH₂—, —CH₂—CH₂—, —CH₂═CH₂—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are illustrative.

Also to be mentioned as aa² are β-amino acids of formula (XXVI).

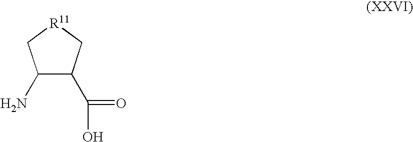

wherein R¹¹ is as previously defined.

In embodiments, aa² is a residue of an N-substituted imino acid or β-amino acid.

It will be appreciated from the above that a very preferred class of products consists of those in which aa¹-aa² is Phe-Pro. In another preferred class, aa¹-aa² is Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula —(CH₂)ₛ-Z. Integer s is 2, 3 or 4 and W is —OH, —OMe, —OEt or halogen (F, Cl, I or, preferably, Br). Particularly illustrative Z groups are —OMe and —OEt, especially —OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

Accordingly, a specific class of acids are those of the formula X-Phe-Pro-Mpg-B(OH)₂, especially Cbz-Phe-Pro-Mpg-B(OH)₂; also included are analogues of these compounds in which Mpg is replaced by a residue with another of the R¹ groups and/or Phe is replaced by Dpa or another aa¹ residue. Also included are compounds in which Cbz is replaced by benzylmethylcarbonyl (Ph-Et-CO—).

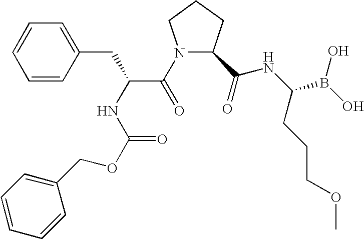

(R,S,R)-TRI 50c Cbz-(R)-Phe-(S)-Pro-(R)-Mpg-B(OH)₂

The aa¹ moiety of the acid is preferably of R configuration. The aa² moiety is preferably of (S)-configuration. Particularly preferred compounds have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of salts of Cbz-Phe-Pro-BoroMpg-OH:

In preferred embodiments, the various aspects of the disclosure relate to pharmaceutically acceptable base addition salts of the described acids.

The disclosure includes base addition salts of Cbz-(R)-Phe-(S)-Pro-(R)-boroMpg-OH (and of other compounds of the formula X—(R)-Phe-(S)-Pro-(R)-boroMpg-OH) which are at least 90% pure, e.g. at least 95% pure.

In broad terms, the base addition salts described herein may be considered to correspond to reaction products of an organoboronic acid as described above with a strong base, e.g. a basic metal compound; the salts are however not limited to products resulting from such a reaction and may be obtained by alternative routes.

The base addition salts are therefore obtainable by contacting a boronic acid disclosed herein with a strong base. The disclosure thus contemplates products (compositions of matter) having the characteristics of a reaction product of an acid of formula (I) and a strong base. The base is pharmaceutically acceptable.

As suitable salts may be mentioned salts of metals, e.g. of monovalent or divalent metals, and stronger organic bases, for example:

1. Alkali metal salts;
2. Divalent, e.g. alkaline earth metal, salts;
3. Group III metals;
4. Salts of strongly basic organic nitrogen-containing compounds, including:
4A. Salts of guanidines and their analogues;
4B. Salts of strongly basic amine, examples of which include (i) aminosugars and (ii) other amines.

Of the above salts, particularly illustrative are alkali metals, especially Na and Li. Also illustrative are aminosugars.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

The disclosure includes therefore products (compositions of matter) which comprise salts which may be represented by formula (V):

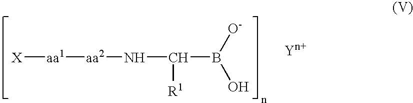

where Yⁿ⁺ is a pharmaceutically acceptable cation obtainable from a strong base, and aa¹, aa², X and R¹ are as defined above. Also included are products in which R¹ is replaced by another R⁹ group.

One class of salts have a solubility of about 10 mM or more, e.g. of at least about 20 mM, when their solubility is determined as described in the examples at a dissolution of 25 mg/ml. More particularly yet they have a solubility of least 50 mM when their solubility is determined as described in the examples at a dissolution of 50 mg/ml.

The disclosure includes salts of boronic acids (I) having an observed stoichiometry consistent with the salt being of (being representable by) the formula “(boronate⁻)ₙ cationⁿ⁺”. One class of such salts are represented by the formula:

[Cbz-(R)-Phe-(S)-Pro-(R)-Mpg-B(OH)(O⁻)]M⁺

where M⁺ represents a monovalent cation, especially an alkali metal cation. It will be understood that the above representation is a notional representation of a product whose observed stoichiometry is unlikely to be literally and exactly 1:1. In any event, a particular salt is Cbz-(R)-Phe-(S)-Pro-(R)-Mpg-B(OH)₂ monosodium salt (TGN 255). In the above formula, the trigonally-represented boronate represents, as always, boronates which are trigonal, tetrahedral or mixed trigonal/tetrahedral.

Particularly exemplary are products which comprise:

(i) species selected from (a) acids of formula (VIII): X—(R)-Phe-(S)-Pro-(R)-Mpg-B(OH)₂ where X is H or an amino-protecting group, especially Cbz, (b) boronate anions thereof, and (c) any equilibrium form of the aforegoing (e.g. an anhydride); and

(ii) ions having a valency n in combination with said species, the species and said ions having an observed stoichiometry consistent with a notional species:ion stoichiometry of n:1. In one class of salts, n is 1.

Considering the counter-ions in turn:

### 1. Monovalent Metal, Especially Alkali Metal Salts

Suitable alkali metals include lithium, sodium and potassium. All of these are remarkably soluble. Lithium and sodium are illustrative because of their high solubility. The lithium and particularly sodium salts are of surprisingly high solubility in relation to potassium amongst others. Sodium is most used in many instances. Salts containing mixtures of alkali metals are contemplated by the disclosure.

The disclosure includes products comprising salts of the formula (VI)

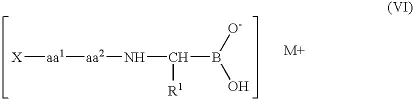

where M⁺ is an alkali metal ion and aa¹, aa², X and R¹ are as defined above, as well as salts in which both hydroxy groups of the boronate group are in salt form (preferably with another identical M⁺ group) and mixtures of such salts. Included also are products wherein R¹ is replaced by another R⁹ group.
2. Divalent, e.g. Alkaline Earth Metal (Group II Metal) Salts

One example of a divalent metal is calcium. Another suitable divalent metal is magnesium. Also contemplated is zinc. The divalent metals are usually used in a boronic acid:metal ratio of substantially 2:1, in order to achieve the preferred monovalent boronate moiety. Salts containing mixtures of divalent metals, e.g. mixtures of alkaline earth metals, are also contemplated.

Further disclosed are products (compositions of matter) which comprise salts which may be represented by the formula (VII):

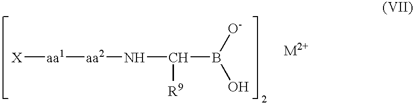

where M²⁺ is a divalent metal cation, e.g. an alkaline earth metal or zinc cation, and aa¹, aa², X and R⁹ are as defined above, as well as salts in which both hydroxy groups of the boronate group are deprotonated and mixtures of such salts. As previously indicated, the boronate may comprise a tetrahedral species.

### 3. Group III Metals

Suitable Group III metals include aluminium and gallium. Salts containing mixtures of Group III metals are also contemplated.

The disclosure includes products comprising salts of the formula (VIII):

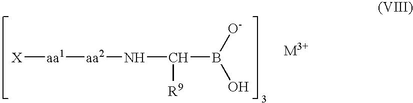

where M³⁺ is a Group III metal ion and aa¹, aa², X and R⁹ are as defined above, as well as salts in which both hydroxy groups of the boronate group are in salt form and mixtures of such salts. As previously indicated, the boronate may comprise a tetrahedral species.

### 4. Strongly Basic Organic Nitrogen-Containing Compounds

The disclosure includes products obtainable by (having the characteristics of a product obtained by) reaction of a peptide boronic acid as defined above and a strong organic base. Two illustrative classes of organic base are described in sections 4A and 4B below. Particularly preferred are acid salts (in which one of the two boronic —OH groups is deprotonated). Most commonly, the salts contain a single type of organic counter-ion (disregarding trace contaminants) but the disclosure contemplates salts containing mixtures of organic counter-ions; in one sub-class, the different counter-ions all fall within the section 4A family described below or, as the case may be, in the section 2B family below; in another subclass, the salts comprise a mixture of organic counter-ions which are not all from the same family (4A or 4B).

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base.

4A. Guanidines and their Analogues

The guanidino compound (guanidine) may in principle be any soluble and pharmaceutically acceptable compound having a guanidino or a substituted guanidino group, or a substituted or unsubstituted guanidine analogue. Suitable substituents include aryl (e.g. phenyl), alkyl or alkyl interrupted by an ether or thioether linkage and, in any event, typically contain from 1 to 6 and especially 1, 2, 3, or 4 carbon atoms, as in the case of methyl or ethyl. The guanidino group may have 1, 2, 3 or 4 substituent groups but more usually has 1 or 2 substituent groups, for instance on a terminal nitrogen. One class of guanidines is monoalkylated; another class is dialkylated. As guanidine analogues may be mentioned thioguanidines and 2-amino pyridines. Compounds having unsubstituted guanidino groups, for example guanidine and arginine, form one particular class.

Salts containing mixtures of guanidines are contemplated by the disclosure.

A particular guanidino compound is L-arginine or an L-arginine analogue, for example D-arginine, or the D- or, preferably, L-isomers of homoarginine or agmatine [(4-aminobutyl)guanidine]. Less preferred arginine analogues are NG-nitro-L-arginine methyl ester, for example, and constrained guanidine analogues, particularly 2-amino pyrimidines, for example 2,6-quinazolinediamines such as 5,6,7,8-tetrahydro-2,6-quinazolinediamine, for example. The guanidino compound may also be a peptide, for example a dipeptide, containing arginine; one such dipeptide is L-tyrosyl-L-arginine.

Some particular guanidino compounds are compounds of formula (VII):

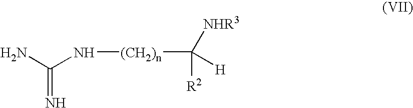

where n is from 1 to 6 and for example at least 2, e.g. 3 or more, and in many instances no more than 5. Most particularly, n is 3, 4 or 5. R² is H or carboxylate or derivatised carboxylate, for example to form an ester (e.g. a C₁-C₄ alkyl ester) or amide. R³ is H, C₁-C₄ alkyl or a residue of a natural or unnatural amino acid (e.g. tyrosine). The compounds of formula (IV) are usually of L-configuration. The compounds of formula (IV) are arginine (n=3; R²=carboxyl; R³═H) and arginine derivatives or analogues.

The disclosure includes products comprising salts of the formula (IX)

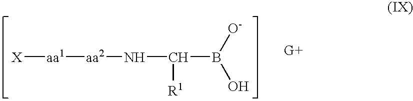

where aa¹, aa², X and R¹ are as defined previously and G⁺ is the protonated form of a pharmaceutically acceptable organic compound comprising a guanidino group or an analogue thereof, as well as salts in which both hydroxy groups of the boronate group are in salt form (preferably with another identical G+group) and mixtures of such salts. Also included are products wherein R¹ is replaced by another R⁹ group.

### 4B. Strongly Basic Amines

The disclosure includes products obtainable by (having the characteristics of a product obtained by) reaction of a peptide boronic acid as defined above and a strong organic base which is an amine. The amine may in principle be any soluble and pharmaceutically acceptable amine.

It is envisaged that a desirable class of amine includes those having polar functional groups in addition to a single amine group, as such compounds will be more hydrophilic and thus more soluble than others. In certain salts, the or each additional functional group is hydroxy. Some amines have 1, 2, 3, 4, 5 or 6 additional functional groups, especially hydroxy groups. In one illustrative class of amines the ratio of (amino plus hydroxy groups):carbon atoms is from 1:2 to 1:1, the latter ratio being particularly preferred. These amines with one or more additional polar functional groups may be a hydrocarbon, especially an alkane, substituted by the amino group and the additional polar group(s). The amino group may be substituted or unsubstituted and, excluding amino substituents, the polar base may contain, for example, up to 10 carbon atoms; usually there are no less than three such carbon atoms, e.g. 4, 5 or 6. Aminosugars are included in this category of polar bases.

The disclosure includes products comprising salts of the formula (X)

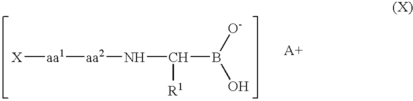

where aa¹, aa², X and R¹ are as defined previously and A⁺ is the protonated form of a pharmaceutically acceptable amine, as well as salts in which both hydroxy groups of the boronate group are in salt form (preferably with another identical A⁺ group) and mixtures of such salts. In one class of such products, A⁺ is the protonated form of an amine described in section 2B(i) below; in another class A⁺ is the protonated form of an amine described in 2B(ii) below. Also included are products in which R¹ is replaced by another R⁹ group.

Two illustrative classes of amine base are described in sections 4B(i) and 4B(ii) below. Particularly preferred are acid salts (in which one of the two boronic —OH groups is deprotonated). Most commonly, the salts contain a single type of amine counter-ion (disregarding trace contaminants) but the disclosure contemplates salts containing mixtures of amine counter-ions; in one sub-class, the different counter-ions all fall within the sub-section 4B(i) family described below or, as the case may be, in the sub-section 4B(ii) family below; in another subclass, the salts comprise a mixture of organic counter-ions which are not all from the same family (4B(i) or 4B(ii)).

4B(i) Aminosugars
The identity of the aminosugar is not critical. Preferred aminosugars include ring-opened sugars, especially glucamines. Cyclic aminosugars are also envisaged as useful. One class of the aminosugars is N-unsubstituted and another, preferred, class is N-substituted by one or two N-substituents (e.g. one). Suitable substituents are hydrocarbyl groups, for example

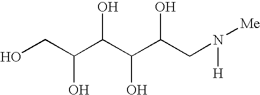

and without limitation containing from 1 to 12 carbon atoms; the substituents may comprise alkyl or aryl moieties or both. Exemplary substituents are C₁, C₂, C₃, C₄, C₅, C₆, C₇ and C₈ alkyl groups, in particular methyl and ethyl, of which methyl is illustrative. Data indicate that aminosugars, especially N-methyl-D-glucamine, are of surprisingly high solubility. A most preferred aminosugar is N-methyl-D-glucamine:

4B(ii) Other Amines
Other suitable amines include amino acids (whether naturally occurring or not) whose side chain is substituted by an amino group, especially lysine.
Some amines are compounds of formula (XI):

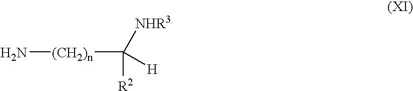

where n, R² and R³ are as defined in relation to formula (IV). The compounds of formula (VI) are usually of L-configuration. The compounds of formula (VI) are lysine (n=4; R²=carboxyl; R³═H) and lysine derivatives or analogues. A most preferred amine is L-lysine.
Other suitable amines are nitrogen-containing heterocycles. At least usually, such heterocyclic compounds are alicyclic; one class of the heterocyclic compounds is N-substituted and another, preferred, class is N-unsubstituted. The heterocycles may contain 6 ring-forming atoms, as in the cases of piperidine, piperazine and morpholine. One class of amines includes N-containing heterocycles substituted by polar substituents, especially hydroxy, e.g. 1, 2 or 3 times.
The disclosure therefore includes amines other than aminosugars which have one or more (e.g. 1, 2, 3, 4, 5 or 6) polar substituents, especially hydroxy, in addition to one amine group. Such compounds may have a ratio of (amino plus hydroxy groups):carbon atoms of 1:2 to 1:1, the latter ratio being particularly preferred.

The disclosure includes mixed salts, i.e. salts containing a mixture of boropeptide moieties and/or counterions but single salts are preferred.

The salts in solid form may contain a solvent, e.g. water. There are included a class of products in which the salts are essentially anhydrous. Also included is a class in which the salts are hydrates.

### Novel Boronic Acids

The disclosure provides novel boronic acids and derivatives thereof, useful in the treatment, e.g. prevention, of thrombosis. The novel acids include those of the formula (IA):

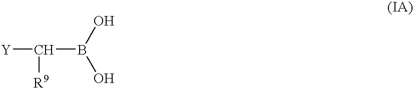

wherein
Y comprises a moiety which, together with the fragment —CH(R⁹)—B(OH)₂, has affinity for the substrate binding site of thrombin and which includes a thrombin P2 domain which comprises a residue of a β-amino acid; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms.

The β-amino acid has affinity for the S2 subsite of thrombin and may be a β-amino acid or a β-amino acid.

The β-amino acid may be the β-amino acid analogue of Gly (i.e. H₂N—CH₂—CH₂—COOH) N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

The disclosure includes a class of compounds in which the β-amino acid is a residue of a β-amino acid having a 4 to 6 membered carbocyclic ring which optionally has one carbon atom replaced by a sulfur and of which the ring-forming carbon atoms include the carbon atoms α- and β- to the carboxyl group (i.e. the β-amino acid comprises a 4 to 6 membered carbocyclic ring which is 1-substituted by carboxyl and 2-substituted by amino and which may at one other position contain an S atom).

Also to be mentioned as the β-amino acid are β-amino acids of formula (XXVI):

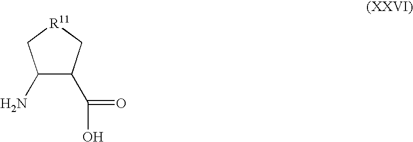

wherein R¹¹ is as previously defined.

In embodiments, the β-amino acid is a residue of an N-substituted imino acid or N-substituted β-amino acid.

Included in the disclosure are acids of formula (II) above in which aa² is a β-amino acid as disclosed herein.

The novel acids may be in the form of the acid, a salt, a prodrug or a salt of a prodrug, as disclosed herein in relation to the formulations. They may be an ester, a base addition salt or an acid addition salt, for example. The ester may be of a diol which has previously been mentioned, e.g. pinacol, pinanediol or a sugar, e.g. mannitol or sorbitol.

The acids and their derivatives may be presented as pharmaceutical formulation, either alone or in combination with a pharmaceutically acceptable diluent, excipient or carrier. The disclosure is not restricted as to the type of formulation, it may be for oral administration, e.g. as a tablet, capsule, granules or powder. The formulation may be a reconstitutable formulation as described herein but it does not need to be. Tablets or capsules may be enterically coated or not.

As parenteral formulations may be mentioned intravenous formulations (e.g. isotonic solutions) or powders/granules for reconstitution as a liquid intravenous formulation. Parenteral formulations may comprise finely divided powder, e.g. freeze dried powder, optionally including a suitable excipient, e.g. isotonic agent.

The compounds may be administered to inhibit thrombin in the treatment of disease, e.g. for an indication described in this specification or any other indication for which thrombin inhibition is beneficial.

Other structural and functional characteristics of embodiments of the new compounds are as described herein in relation to the compounds used in the formulations, methods and uses disclosed herein.

The novel compounds provide a choice. It is contemplated that, at least in embodiments, the compounds will, at least in broad terms, maintain or improve potency or specificity, or both as compared with TRI 50c.

At least in embodiments, the compounds may maintain or enhance bioavailability. Other properties of novel compounds which may lend them pharmaceutical usefulness may include storage stability or ease of formulation, for example.

The synthesis of 2-amino-cycloalkylcarboxylic acids is described in WO 98/03540.

### Synthetic Methods and Their Products

### 1. Peptide/Peptidomimetic Synthesis

The synthesis of boropeptides, including, for example, Cbz-D-Phe-Pro-BoroMpg-OPinacol is familiar to those skilled in the art and described in the prior art mentioned above, including Claeson et al (U.S. Pat. No. 5,574,014 and others) and Kakkar et al (WO 92/07869 and family members including U.S. Pat. No. 5,648,338). It is described also by Elgendy et al Adv. Exp. Med. Biol (USA) 340:173-178, 1993; Claeson, G. et al Biochem. J. 290:309-312, 1993; Deadman et al J. Enzyme Inhibition 9:29-41, 1995, and by Deadman et al. J. Med. Chem. 38:1511-1522, 1995.

Stereoselective synthesis with S or R configuration at the chiral B-terminal carbon may be conducted using established methodology (Elgendy et al Tetrahedron. Lett. 33:4209-4212, 1992; WO 92/07869 and family members including U.S. Pat. No. 5,648,338) using (+) or (−)-pinanediol as the chiral director (Matteson et al J. Am. Chem. Soc. 108:810-819, 1986; Matteson et al Organometallics. 3:1284-1288, 1984). Another approach is to resolve the requisite aminoboronate intermediate (e.g. Mpg-BOPinacol) to selectively obtain the desired (R)-isomer and couple it to the dipeptide moiety (e.g. Cbz-(R)-Phe-(S)-Pro, which is the same as Cbz-D-Phe-L-Pro) which will form the remainder of the molecule.

The boropeptides may be synthesised initially in the form of boronic acid esters, particularly esters with diols. Such diol esters may be converted to the peptide boronic acid as described next.

### 2. Ester to Acid Conversion

A peptide boronate ester such as Cbz-(R)-Phe-Pro-BoroMpg-OPinacol may be hydrolysed to form the corresponding acid.

A novel technique for converting a diol ester of a peptide boronic acid of for example, formula (I) into the acid comprises dissolving the diol ester in an ether and particularly a dialkyl ether, reacting the thus-dissolved diol with a diolamine, for example a dialkanolamine, to form a product precipitate, recovering the precipitate, dissolving it in a polar organic solvent and reacting the thus-dissolved product with an aqueous medium, e.g. an aqueous acid, to form the peptide boronic acid. The boronic acid may be recovered from the organic layer of the mixture resulting from the reaction, for example by removing the solvent, e.g. by evaporation under vacuum or distillation. The reaction between the diol ester and the diolamine may be carried out under reflux, for example.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The alkyl groups of the dialkyl ether preferably have 1, 2, 3 or 4 carbon atoms and the alkyl groups may be the same or different. An exemplary ether is diethyl ether.

The alkyl groups of the dialkanolamine preferably have 1, 2, 3 or 4 carbon atoms and the alkyl groups may be the same or different. An exemplary dialkanolamine is diethanolamine. The diethanolamine/boronic acid reaction product hydrolyses in water at room temperature and the rate of hydrolysis may be accelerated by adding acid or base.

The polar organic solvent is preferably CHCl₃. Other examples are polyhalogenated alkanes generally and ethyl acetate. In principle, any polar organic solvent is acceptable other than alcohols.

The aqueous acid is suitably a strong inorganic acid at a pH in the region of 1 such as hydrochloric acid, for example.

After reaction with the acid, the reaction mixture is suitably washed with, for example, NH₄Cl or another mild base.

An example of a specific procedure is as follows

1. The pinacol or pinanediol ester of the selected peptide boronic acid is dissolved in diethylether.
2. Diethanolamine is added and the mixture is refluxed at 40° C.
3. The precipitated product is removed (filtered), washed (usually several times) with diethyl ether or another polar organic solvent other than an alcohol, and dried (e.g. by evaporation under vacuum).
4. The dry product is dissolved in a polar organic solvent other than an alcohol, e.g. CHCl₃. Aqueous acid or base is added, e.g. hydrochloric acid (pH 1), and the mixture is stirred for e.g. approximately 1 h at room temperature.
5. The organic layer is removed and washed with NH₄Cl solution.
6. The organic solvent is distilled off and the residual solid product is dried.

The above process results in the formation of what may conveniently be referred to as a “diolamine adduct” of the peptide boronic acid, especially such adducts with diethanolamine, and such adducts are themselves included in the disclosure.

It will be appreciated that the aforegoing technique comprises an example of a method for recovering an organoboronic acid product, the method comprising providing in a solvent a dissolved mixture comprising the organoboronic acid in a soluble form and a compound having two hydroxy groups and an amino group (i.e. a diolamine), causing or allowing the organoboronic acid and the diolamine to react to form a precipitate, and recovering the precipitate. The soluble form of the organoboronic acid may be a diol ester, as discussed above. The solvent may be an ether, as discussed above. The organoboronic acid may be one of the organoboronic acids referred to in this specification, for example it may be of Formula (I) or (III). The method described in this paragraph is novel and forms an aspect of the disclosure. A recovery method is filtration.

The reaction between the diolamine and the soluble form of the organoboronic acid is suitable carried out at an elevated temperature, for example under reflux.

Another aspect of the disclosure is a method for recovering an organoboron species, comprising
providing, in a form soluble in an ether, an organoboronic acid, for example a drug such as, e.g., a compound of formula (III);
forming a solution of the soluble form in the ether;
combining the solution with a dialkanolamine and allowing or causing the dialkanolamine to react with the soluble form of the organoboronic acid to form an insoluble precipitate; and
recovering the precipitate.

The term “soluble” in the preceding paragraph refers to species which are substantially more soluble in the reaction medium than is the precipitated product. In variants of the method, the ether is replaced by toluene or another aromatic solvent.

The diethanolamine precipitation technique described above is an example of another novel method, which is a method for recovering from ether solution a pinacol or pinanediol ester of a peptide boronic acid, comprising dissolving diethanolamine in the solution, allowing or causing a precipitate to form and recovering the precipitate. The disclosure encompasses variants of this methods in which another diol than pinacol or pinanediol is used.

The precipitated material, i.e. the “adduct”, may be converted into the free organoboronic acid, for example by contacting it with an acid. The acid may be an aqueous acid, for example an aqueous inorganic acid, e.g. as described above. The precipitate may be dissolved, for example in an organic solvent, prior to being contacted with the acid.

The disclosure therefore provides a method for making an organoboronic acid, comprising converting its diolamine reaction product to the acid.

The acid resulting from the methods described in the previous two paragraphs may be converted to a salt of the acid with a multivalent metal, which salt may in turn be formulated into a pharmaceutical composition in parenteral dosage form.

### 3. Salt Synthesis

### 3.1 Base Addition Salts

In general, the salts may be prepared by contacting the relevant peptide boronic acid with a strong base appropriate to form the desired salt. In the case of metal salts, the metal hydroxides are suitable bases (alternatively, metal carbonates might be used, for example), whilst sometimes it is more convenient to contact the acid with a relevant metal alkoxide (e.g. methoxide), for which purpose the corresponding alkanol is a suitable solvent. Salts with organic bases may be prepared by contacting the peptide boronic acid with the organic base itself. Illustrative salts are acid salts (one —BOH proton replaced) and, to make acid salts with a monovalent cation, the acid and the base are suitably reacted in substantially equimolar quantities. Generally stated, therefore, the usual acid:base molar ratio is substantially n:1, where n is the valency of the cation of the base.

In one procedure, a solution of the peptide boronic acid in a water-miscible organic solvent, for example acetonitrile or an alcohol (e.g. ethanol, methanol, a propanol, for example iso-propanol, or another alkanol), is combined with an aqueous solution of the base. The acid and the base are allowed to react and the salt is recovered. The reaction is typically carried out at ambient temperature (e.g. at a temperature of from 15 to 30° C., e.g. 15 to 25° C.), but an elevated temperature may be used, for example up to the boiling point of the reaction mixture but more usually lower, e.g. a temperature of up to 40° C. or 50° C. The reaction mixture may be allowed to stand or be agitated (usually stirred).

The time during which the acid and the base are allowed to react is not critical but it has been found desirable to maintain the reaction mixture for at least one hour. A period of from one to two hours is usually suitable but longer reaction times may be employed.

The salt may be recovered from the reaction mixture by any suitable method, for example evaporation or precipitation. Precipitation may be carried out by adding an excess of a miscible solvent in which the salt has limited solubility. In one preferred technique, the salt is recovered by evacuating the reaction mixture to dryness. The salt is preferably thereafter purified, for example by redissolving the salt before filtering the resulting solution and drying it, for example by evacuating it to dryness. The redissolution may be performed using water, e.g. distilled water. The salt may then be further purified, for example in order to remove residual water by further redissolution in a suitable solvent, which is advantageously ethyl acetate or THF followed by evaporating to dryness. The purification procedure may be carried out at ambient temperature (say, 15 to 30° C., e.g. 15 to 25° C.), or at a modestly elevated temperature, such as e.g. a temperature not exceeding 40° C. or 50° C.; for example the salt may be dissolved in water and/or solvent by agitating with or without warming to, for example, 37° C.

Also included is a method for drying the salts of the disclosure and other peptide boronic acid salts, comprising dissolving them in an organic solvent, e.g. ethyl acetate or THF, and then evaporating to dryness, e.g. by evacuation.

Generally, preferred solvents for use in purifying the salts are ethyl acetate or THF, or perhaps another organic solvent.

A general procedure for synthesising salts of Cbz-Phe-Pro-BoroMpg-OH is as follows:

Cbz-Phe-Pro-BoroMpg-OH (20.00 g, 38.1 mM) is dissolved in acetonitrile (200 ml) with stirring at room temperature. To this solution is added the requisite base in solution in distilled water (190 ml); the base is added as a 0.2M solution for a monovalent cation. The resultant clear solution is allowed to react for example by being left to stand or being agitated, for a usual period, in either case, of from one to two hours. The reaction is typically carried out at ambient temperature (e.g. 15-30° C., e.g. 15 to 25° C.) but alternatively the temperature may be elevated (e.g. up to 30° C., 40° C. or 50° C.). The reaction mixture is then evacuated to dryness under vacuum with its temperature not exceeding 37° C., typically to yield a white brittle solid or an oil/tacky liquid. The oil/tacky liquid is redissolved in the minimum amount of distilled water necessary (200 ml to 4 L), typically with warming (e.g. to 30-40° C.), usually for up to 2 hours. The solution is filtered, suitably through filter paper, and evacuated to dryness, again with the temperature of the solution not exceeding 37° C., or freeze dried. The resultant product is dried under vacuum overnight to normally yield a white brittle solid. If the product is present as an oil or tacky solid then it is dissolved in ethyl acetate and evacuated to dryness to produce the product as a white solid. The white solid is typically a coarse, amorphous powder.

In variations of the aforegoing general procedure, the acetonitrile is replaced by another water-miscible organic solvent, notably an alcohol, as discussed above, especially ethanol, methanol, iso-propanol or another propanol.

Where a boronic acid salt is less soluble in a selected reaction medium for salt formation such that its direct preparation from the corresponding acid and base is inconvenient, the less soluble salt may be prepared from a salt more soluble in the reaction medium.

There is provided also the use of a boronic acid to make a base addition salt of the disclosure. Included also is a method of preparing a product of the disclosure, comprising contacting a boronic acid, e.g. of formula (I), (II) or (III), with a base capable of making such a salt.

### 3.2 Acid Addition Salts

In general, the acid addition salts may be prepared by contacting the relevant boropeptide (e.g. boronic acid or ester or other prodrug) with an acid appropriate to form the desired salt.

### 4. Separation of Stereoisomers

The stereoisomers of a peptide boronic ester or a synthetic intermediate aminoboronate may be resolved in, for example, any known way. In particular, stereoisomers of boronic esters may be resolved by HPLC.

### 5. “High Purity” Synthesis

The literature teaches that organoboronic acids are degraded by oxidation of the C—B bond. See for example Wu et al (see above). Earlier work on the salts of TRI 50c confirmed that these salts and/or intermediates in their preparation are slightly unstable, to the extent that the salts were found to contain a boron-free impurity, designated impurity I, which was evidently generated by C—B bond cleavage. The salts as a class are significantly more stable to such degradation than the free acid.

These earlier TRI 50c salts were made via the general methods described in Examples 5 and 9 of this specification. Impurity I has the following structure:

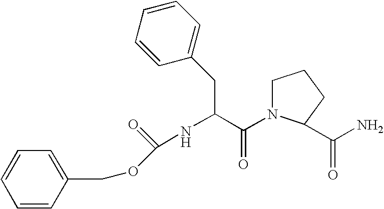

Relative chiral purity of salts made following the general procedure of Examples 5 and 9 was achieved by resolving by HPLC the pinacol ester of TRI 50c, designated TRI 50b, and converting the thus-resolved TRI 50b into the salts. Such an HPLC procedure is not acceptable for normal commercial drug production.

It has further been found that the prior art synthesis summarised earlier under the heading “Aminoboronate Procedure” results, when applied to the synthesis of TRI 50c or an ester thereof, in formation of an impurity designated Impurity IV:

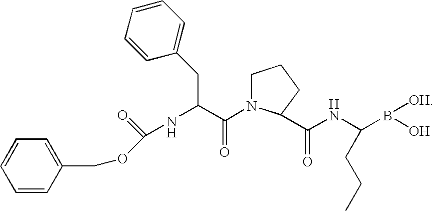

Attempts to separate Impurity IV from TRI 50c have not succeeded. The same applies to TRI 50c salts and esters and the corresponding salts and esters of Impurity IV. No purification technique which has been tried can prevent the presence of Impurity IV if said prior art synthesis is used.

Amongst other things, the synthetic methods described in this section of the specification addresses the problems of controlling C—B bond cleavage in organoboronic compounds as well as providing chirally purified salts of TRI 50c and other organoboronic acids on a commercial scale. In this regard, it has been found that C—B bonds seem to be cleaved by a non-oxidative mechanism which occurs in the presence of many solvents, including water and e.g. aqueous acids and bases, amongst others.

Chirally-selective precipitation may be used to recover organoboronic acids in high purity.

Thus C—B bond cleavage (and hence in particular generation of Impurity I) may be controlled by:
Selection of acetonitrile as a solvent, where a solvent is required in processing and acetonitrile has the necessary solvation power; in particular acetonitrile is selected in process where a polar solvent is desirable or necessary.
Avoiding excessive contact with water.

In terms of TRI 50c salt production, therefore, the disclosure includes processes comprising one, two or three of the following features:
(i) resolution of the (R,S,S) and (R,S,R) epimers of TRI 50c by chirally selective precipitation using diethanolamine and conveniently, but not necessarily, using as starting material TRI 50c in the form of an ester, for example the pinacol ester;
(ii) control of the duration and/or conditions of hydrolysis of TRI 50c diethanolamine ester, for example as obtained by such precipitation, to control C—B bond breakage;
(iii) use of acetonitrile as solvent for TRI 50c, for example as obtained by such hydrolysis, for the purposes of reacting the TRI 50c with a base to form the salt. Another favourable solvent can be tetrahydrofuran.

As an optional, or even stand-alone, fourth feature, TRI 50c salts may be dried by azeodrying using acetonitrile.

It is considered that C—B bond cleavage may occur by a nucleophilic mechanism, and the disclosure therefore includes methods in which opportunities for nucleophilic attack are minimised.

The above four features, or any one, two or three of them, may be applied to the manufacture and processing of other boronic compounds, particularly acids of formula (I) and their derivatives (e.g. esters and salts).

The disclosure provides in one aspect, therefore, the use of diethanolamine to resolve by selective precipitation the diastereomers of boronic acids of formula (I). The starting material may be an acid (I) or a derivative thereof capable of forming a diethanolamine ester of the boronic acid. The precipitation selects acids having a chiral centre C* of (R) configuration as precipitate. The precipitate may be recovered and converted to the corresponding boronic acid or a salt thereof. The salt may be made into a pharmaceutical formulation.

For optimised chiral purity and yield, the diethanolamine may be used in an amount of about 1.25±0.1 equivalents based on initial equivalents of boronic acid having a chiral centre C* of (R) configuration.

The initial boronic acid or acid derivative may for example comprise from 50% to 60% molecules having chiral centre C* of (R)-configuration and from 40% to 50% molecules having chiral centre C* of (S)-configuration.

The method opens the way to commercialisation of the boronic acids (I) and their derivatives, particularly salts, as pharmaceuticals. Commercial scale products and activities using the boronic acids (I) and their derivatives are therefore provided.

In one embodiment, there is provided a process for separating diastereomers of a boronic acid of formula (I), comprising:
combining in diethylether solution (A) a boronic species selected from the boronic acid (I) and its esters, the boronic species including molecules having a chiral centre C* of (R) configuration and molecules having a chiral centre C* of (S) configuration, and (B) diethanolamine, the diethanolamine being in an amount of about 1.25±0.1 equivalents based on the boronic species in which the chiral centre C* is of (R) configuration, and mixing to form a mixture;
causing or allowing the boronic species and the diethanolamine to react until a precipitate forms; and
recovering the precipitate.

When the starting material is an ester, it may be an ester of the boronic acid with an alcohol selected from the group consisting of alcohols whose sole potential electron donor heteroatoms are oxygens which, in the boronic ester, correspond to the oxygens of the ester functional group.

In some methods, the diethanolamine is in an amount of from 1.2 to 1.3 equivalents based on the boronic species in which chiral centre C* is of (R) configuration.

There are included processes in which the boronate species is an ester of the boronic acid and a diol, in particular a diol which is not sterically hindered. As exemplary diols may be mentioned pinacol, neopentylglycol, 1,2-ethanediol, 1,2-propanediol, 1,3-propanediol, 2,3-butanediol, 1,2-diisopropylethanediol, or 5,6-decanediol. A particular diol is pinacol.

The boronic species and the diethanolamine may be caused to react by heating the mixture to an elevated temperature, for example the mixture may be refluxed. e.g. for at least 10 hours.

The precipitate may be recovered by filtration. The recovered precipitate may be washed with diethylether. The recovered precipitate, after washing if such takes places, may be dissolved in a solvent selected from CH₂Cl₂ and CHCl₃ and reprecipitated by combining the resulting solution with diethylether. A particular solvent is CH₂Cl₂.

The recovered precipitate may be converted to the acid of formula (I), suitably by hydrolysis, for example by dissolving the precipitate in an organic solvent selected from e.g. halohydrocarbons and combinations thereof, agitating the resulting solution with an aqueous liquid, e.g. an aqueous acid having a pH of below 3, whereby the dissolved precipitate is converted to the formula (I) acid, and recovering the formula (I) acid by evaporation. The organic solvent may be CH₂Cl₂ or CHCl₃. A particular solvent is CH₂Cl₂. In some processes, organic solvent is further evaporated from the recovered formula (I) acid.

The disclosure includes methods in which an ester of a boronic acid (I), particularly a diethanolamine ester, is hydrolysed in a manner which controls C—B bond cleavage. In particular, this involves limiting the period of hydrolysis at the selected temperature. In the case of diethanolamine ester hydrolysis, the hydrolysis is suitably carried out at room temperature, or less, for a period not exceeding about 30 minutes, e.g. not exceeding about 20 minutes, and optimally of about 20 minutes.

Thus the recovered precipitate referred to in the last paragraph but one may be hydrolysed using an aqueous acid, particularly 2% hydrochloric acid or another mineral acid of similar pH, for no more than about 30 minutes at about room temperature, or less. Suitably, the precipitate is dissolved in a non-nucleophilic organic solvent (e.g. a halohydrocarbon or halohydrocarbon mixture for example CH₂Cl₂) and the resulting solution is contacted with the aqueous acid for a period as previously described. The precipitate is thereby hydrolysed to form the free acid of formula (I), which remains in the organic solvent. The organic solvent may be separated from the aqueous medium and then evaporated to obtain solid acid of formula I.

There are included processes in which a formula (I) acid, for example obtained as described in the preceding paragraph, is dried. In a class of processes, the formula (I) acid is dried when it is in the organic solvent by contacting the solvent with a hygroscopic solid.

Included are processes in which the formula (I) acid, when in the organic solvent, is washed with an aqueous ammonium salt.

Chirally purified boronic acid may be converted to a pharmaceutically acceptable base addition salt thereof, in particular by dissolving the acid in acetonitrile, combining the resultant solution with an aqueous solution or suspension of a pharmaceutically acceptable base, and causing or allowing the base and the acid to react, then evaporating to dryness to obtain an evaporation residue. The step of causing or allowing the acid and the base to react may comprise agitating the combination of the acetonitrile solution of the acid and the aqueous solution or suspension of the base at a temperature of not more than 35° C. and often of not more than 30° C., e.g. not more than 25° C.; an optimal temperature is room temperature, in which case a reaction time of about 2 hours might be appropriate. The process may further comprise:
(i) redissolving the evaporation residue in acetonitrile and evaporating the resulting solution to dryness; and
(ii) repeating step (i) as often as necessary to obtain a dry evaporation residue.

In some processes the dry evaporation residue is dissolved in acetonitrile or tetrahydrofuran to form a solution, and the solution is combined with (e.g. slowly added to, at a rate sufficiently slow to avoid lump formation) a 3:1 to 1:3 v/v mixture of diethylether and an aliphatic or cycloaliphatic solvent to form a precipitate, said solution being added to the diethylether/(cyclo)aliphatic solvent mixture in a ratio (solution:mixture) of from 1:5 to 1:15 v/v. The precipitate is recovered and some or substantially all remaining solvent is removed from the recovered precipitate whilst maintaining the temperature at no more than 35° C., e.g. is removed under reduced pressure. Included are processes in which the temperature at the start of the drying process is about 10° C. and is increased during the process to 35° C. The aliphatic or cycloaliphatic solvent may have 6, 7 or 8 carbon atoms; the solvent may be an alkane, for example an n-alkane, e.g. n-heptane. Some reactions may be carried out at ambient temperature, which may e.g. be 15-30° C., e.g. 20-30° C.; sometimes ambient temperature may be room temperature.

The salts produced by the invention may contain a trace amount of the aliphatic or cycloaliphatic solvent, e.g. an amount of less than 0.1%, particularly less than 0.01%, for example an amount of about 0.005%.

In the process for making the salt, the base may comprise a cation of valency n and be used in a stoichiometry (boronic acid:base) of about n:1. In particular processes, the base is an alkali metal or alkaline earth metal base, for example an alkali metal hydroxide or an alkaline earth metal hydroxide. As one base may be mentioned sodium hydroxide. As another base may be mentioned calcium hydroxide. The disclosure includes processes in which the base is sodium hydroxide and the dry evaporation residue is dissolved in acetonitrile. The disclosure includes processes in which the base is calcium hydroxide and the dry evaporation residue is dissolved in tetrahydrofuran.

The disclosure is not limited as to the method by which the boronic acids of Formula (I) are obtained (for example as an ester thereof). However, in one class of subject matter, the Formula (I) acid has an R¹ group of the formula —(CH₂)ₛ—O—R³ in which R³ is methyl or ethyl and s is independently 2, 3 or 4, and the Formula (I) acid is prepared via an intermediate of Formula (XXV):

(HO)₂B—(CH₂)ₛ—O—R³ (XXV),

which intermediate is made by reaction between a borate ester and a suitable 1-metalloalkoxyalkane.

A novel aspect of the disclosure comprises the Formula (XXV) intermediates.

The Formula (XXV) intermediates may be made by reacting a 1-metalloalkoxyalkane, where the alkoxyalkane is of the formula —(CH₂)ₛ—O—R³, with a borate ester to form a compound of Formula (XXV).

It will be appreciated that the above method provides a general procedure for making alkoxyalkylboronic acids, which may be presented by the formula R^{Z}—O—R^{Y}—B(OH)₂. Such alkoxyalkylboronic acids may be converted to aminoboronates, and the aminoboronates may be derivatised at their amino group to form an amide bond linked to another moiety. In other words, the aminoboronates may be converted to boropeptides. The method will now be described further with non-limiting reference to compounds of Formula (XXV).

The starting materials for the reaction may be a metalloalkoxyalkane, e.g. a Grignard reagent, obtainable from 1-haloalkoxyalkane of the formula Hal-(CH₂)ₛ—O—R³ (where Hal is a halogen) and a borate ester. The metal is in particular magnesium. Another metal is lithium, in which case the metallo reagent may be prepared by reacting the 1-haloalkoxyalkane with butyl lithium. Where the method includes preparation of the metallo reagent from the haloalkoxyalkane, the haloalkoxyalkane may be a chloroalkoxyalkane; the corresponding bromo compounds may also be used. To make a Grignard reagent, magnesium may be reacted with the haloalkoxyalkane.

Suitable borate esters are esters of mono- and di-functional alcohols (e.g. of EtOH, MeOH, BuOH, pinacol, glycol, pinanediol etc). For example, the ester may be of the formula B(OR^{a})(OR^{b})(OR^{c}) where R^{a}, R^{b} and R^{c} and C₁-C₄ alkyl and may be the same as each other.

An exemplary procedure for making a Formula (XXV) intermediate, illustrated with reference to methoxypropane as the alkoxyalkane species, is:

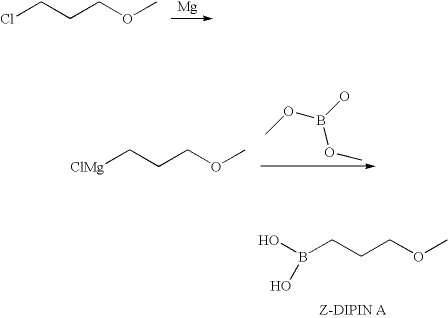

The reactions are suitably carried out in an organic solvent, e.g. THF.

The above-described procedure for making alkoxyalkylboronic acids avoids generation of Impurity IV (see above), or its analogues in those cases where the end product is not TRI 50c or a derivative (salt, ester etc) thereof. The procedure therefore provides a unique route to making TRI 50c, its esters and salts, uncontaminated by Impurity IV, and for making other aminoboronic acids which are substituted α- to the boron by an alkoxyalkyl group and are uncontaminated by impurities analogous to Impurity IV.

An alkoxyalkylboronic acid, i.e. a compound which may be represented by the formula R^{Z}—O—R^{Y}—B(OH)₂, may be converted to an aminoboronic compound, for example a boropeptide, by any suitable procedure, e.g. one known in the art. A reaction scheme for making alkoxyalkylboronic acids into aminoboronates, and for converting aminoboronates into peptide boronates is illustrated with reference to synthesis of TRI 50c at the start of the Examples of this specification. The reaction scheme may be modified as desired, e.g.: diethanolamine precipitation and subsequent steps may be omitted, and/or reagent substitutions may be made. For example, pinacol may be replaced by another diol. LDA is a non-nucleophilic strong base and may be replaced by another such base. Other examples include, but are not limited to, lithium diisopropylamide, lithium 2,2,6,6-tetramethylpiperidine, 1-lithium 4-methylpiperazide, 1,4-dilithium piperazide, lithium bis(trimethylsilyl) amide, sodium bis(trimethylsilyl)amide, potassium bis(trimethylsilyi)amide, isopropyl magnesium chloride, phenyl magnesium chloride, lithium diethylamide, and potassium tert-butoxide. The reactions may be carried out in any suitable solvent: where n-heptane is used in the Examples, it may be replaced by another inert non-polar solvent, e.g. another aliphatic or cycloaliphatic solvent, for example an alkane, e.g. an n-alkane.

It will be appreciated from the aforegoing that the above described methods may be used in the manufacture of organoboronic acids salts as described. It is not necessary for sequential steps to be carried out as one operation or at the same site: they may be performed in this way or different processes (different parts of the overall synthesis) may be distributed in time and/or space. Particular end product salts are monosodium, monolithium, hemicalcium and hemimagnesium salts, for example of TRI 50c.

Generally, the reactions may suitably be carried out with a non-nucleophilic solvent. Where a nucleophilic solvent is present, minimum contact is preferred, for example in the case of hydrolysis of diethanolamine esters.

### 6. High Purity Products

The “high purity products” of the disclosure include inter alia boronic acids, diethanolamine esters and salts obtainable by (having the characteristics of a product obtained by) the disclosed methods. Also included are products obtained directly or indirectly by the disclosed methods. Such products may be formulated for oral administration, and such oral formulations are included in the disclosure.

Particular products of the invention are base addition salts of a boronic acid of formula (I) having the chiral purity of such salt when prepared by a method described herein. Other products are base addition salts of a boronic acid of formula (I) having the purity of such salt when prepared by a method described herein.

Product identities will be apparent from the preceding description and the following examples. In addition, products of the disclosure are described in the claims. Of particular note are the data in Example 38, indicating that the processes of the invention can remarkably achieve end product salts free of impurities detectable by HPLC. In other instances, the salts are substantially free of impurities, e.g. at least 98% pure, more usually at least 99% pure, e.g. at least 99.5% pure, in terms of reverse phase (RP) HPLC percentage peak area. Salts may at least 99.3%, 99.4%, 99.5% 99.6%, 99.7%, 99.8% or 99.9% pure, in terms of reverse phase (RP) HPLC percentage peak area. Suitable RP HPLC procedures comply with reference 1 and/or reference 2 and/or reference 3 of Example 38. Included also are products at least substantially free of Impurity I and analogues, products free of Impurity IV and analogues, and products containing small traces of non-polar solvent, e.g. n-heptane. The trace amount of non-polar solvent may be less than 0.2%, 0.1%, 0.05%, 0.01% or 0.005% as determined by GC-headspace chromatography.

Included also are salts containing less than 410 ppm acetonitrile.

Some salts contain impurities of less than 10,000 ppm, 5000 ppm, 1000 ppm, or 500 ppm.

### The Formulations

Included in the formulations of the disclosure are solid phase products adapted for the active ingredient (the base addition salt of the boronic acid) to be administered orally and be put into solution or suspension before the active principle (the boronic acid and/or its corresponding boronate species) enters the stomach. The active ingredient may be put into solution or suspension prior to administration or, alternatively, in the mouth.

A first class of formulation, therefore, comprises particulate formulations, i.e. powders and granules, for oral administration after reconstitution into a liquid preparation, and preferably into a drinkable preparation. It is generally preferred for the liquid preparation to be a solution rather than a suspension and certain particulate formulations are for dissolving to form a drinking solution. The formulation may be for reconstitution into a suspension or more normally a solution having a volume of from about 50 ml to about 150 ml. The disclosure also contemplates reconstituted volumes of less than about 50 ml or of more than about 150 ml, e.g. of up to about 250 ml and more often of up to about 200 ml. If a small volume of reconstituted solution is required, this can be achieved at least in preferred embodiments, e.g. in the case of the monosodium salt of TRI 50c an amount of salt equivalent to 600 mg TRI 50c may be readily dissolved in 10 ml water to form a pH 9.88 solution.

The disclosure includes particulate formulations adapted for reconstitution with water to form a solution or suspension of the boronic acid salt (or free boronic acid, prodrug or prodrug salt), the solution containing a therapeutically effective amount of the boronic acid salt and having a volume of from about 50 ml to about 150 ml. Such formulations may contain a pharmaceutically acceptable organic acid in an amount sufficient to reduce the pH of the solution, e.g. to a value below 9.5 in the case of salts.

It has been found possible to form surprisingly concentrated boronate salt solutions (of up to about 600 mg/ml in the case of TRI 50c monosodium salt) at a pH of about 9.5. However, a solution with a pH of 9.5 will often be undesirably distasteful to drink. Accordingly, a pharmaceutically acceptable organic acid may be included in the particulate formulation in an amount selected to reduce the pH to a value at which the solution is more palatable but at which a solution of drinkable quantity (e.g. about 50 ml to about 150 ml) may be formed by reconstituting the particulate formulation. As the organic acid may be mentioned citric acid, tartaric acid or malic acid, for example. In many instances, citric acid is chosen.

Experiments have been performed to test the solubility of TRI 50c monosodium salt at different pH values. All the experiments were conducted using a quantity of the salt equivalent to 600 mg TRI 50c free acid. In a first series of experiments, this amount of the salt was dissolved in 50 ml water to form a solution of approximately pH 9.5. Dilute aqueous HCl was added to determine how much the pH could be reduced before precipitation occurred. It was found that the salt tended to precipitate when the pH of the reconstituted solution was reduced below 9 and the pH of a reconstituted liquid having this concentration of salt may therefore be maintained at 9 or more, e.g. 9.2 or more, to keep the salt in solution.

In a second series of experiments, the same amount of the salt was dissolved in 150 ml water, and citric acid was added. It was found that the pH could be reduced to a value of 3.7-3.8 using citric acid before precipitation occurred. In other words if, in the case of a salt dosage equivalent to 600 mg TRI 50c, the patient instructions are to prepare a solution in at least 150 ml water, a quantity of organic acid (e.g. citric acid) can be included in the formulation which will reduce the pH to a value of, say, not less than 4, without a risk of precipitation. Since acid solutions tend to be more palatable than alkaline ones, and citric acid is a common flavouring agent, this behaviour of the salt is highly beneficial. In practical terms, up to 200 mg citric acid may be combined with TRI 50c monosodium salt (600 mg, calculated as TRI 50c) for a preparation to be reconstituted in 150 ml water or more. In general, it is contemplated that the boronate will be formulated to form a reconstituted solution having a pH of from 4 to 8, e.g. 4 to 7, optionally 5 to 6.

Of course, the absolute amount of citric or other acid would be varied with (i) the absolute amount of the salt and (ii) the desired reconstituted volume, in line with the guidance from the above results and such routine experimentation as might be necessary.

TRI 50c sodium salt is a representative of other salts disclosed herein, particularly other alkali metal salts but also other soluble salts or derivatives, such as aminosugar salts or sugar esters. Accordingly, these results indicate that embodiments of the disclosed boronic acid compounds may be formulated with organic acids to make formulations which, optionally when containing additional flavours, will be acceptable as regards flavour.

It is envisaged that therapeutically effective doses of the boronic acid compound (e.g. salt) for a human adult will include dosages of from about 0.2 mole boronate to about 1.5 moles boronate. The dosage may be at least about 0.35 moles. The dosage is usually not more than about 1.5 moles boronate, more usually about 1.35 moles or less, e.g. not more than about 1.25 moles; in some instances it is at most about 1 mole. In one instance, the therapeutically effective dose is from about 0.4 mole to about 0.85 mole boronate.

The formulations may be presented in monodose units, i.e. units containing exactly a single dose of active compound for reconstitution. Thus, in some embodiments, there are provided monodose units containing TRI 50c (or, e.g., its salts) in an amount of from about 100 mg to about 750 mg, e.g. about 100 mg to about 700 mg and more usually of from about 200 mg to about 600 mg, e.g. about 200 mg to about 500 mg, calculated as TRI 50c. A particular class of unit dosage forms contains from about 200 mg to about 450 mg TRI 50c or TRI 50c salt, calculated as TRI 50c, e.g. about 300 mg; the salt is suitably a mono alkali metal, e.g. sodium, salt. In place of the salt, another soluble form of TRI 50c may be used, e.g. a sugar derivative.

The aforesaid weights of TRI 50c salts translate into moles as follows:

| Weight (calc. as TRI 50c) | Moles TRI 50c |
|---|---|
| 100 mg | 0.19 |
| 200 mg | 0.38 |
| 450 mg | 0.86 |
| 500 mg | 0.95 |
| 600 mg | 1.14 |
| 700 mg | 1.33 |
| 750 mg | 1.43 |

The granules or powder will typically contain an anti-microbial preservative and a flavouring, both normally being distributed throughout the formulation. Less frequently, the granules or powder will contain just one of these two components. Additionally, or less frequently alternatively, they may contain one or more other excipients as described above under the heading “Oral Dosage Forms”. In some embodiments, a powder or granular formulation may include an effervescent system, e.g. as previously described in relation to effervescent tablets.

The granules or powder will be filled into a container after their manufacture. Usually, the container is suitable for dispensing the contents into a drinking vessel containing water (which may be flavoured or unflavoured, or replaced by fruit juice or another beverage product). The container will in most instances contain a unit of the formulation for dispensing. It may be a monodose container or, for flexibility, the formulation may be presented as a divided dose, in which case the container will include a fraction of envisaged doses and a desired number of containers is selected to make up a single dose for any individual patient.

Suitably, the unit container form is a sachet or a plastics container, e.g. having a moulded plastics body. As an alternative to single-unit containers, there may be mentioned metered dose containers having a reservoir of the formulation and a metering device to enable an appropriate dosage of the formulation to be dispensed.

The disclosure includes oral formulations of the described boronic acid salts in the form of effervescent tablets for combining with a liquid to form an orally administrable solution or suspension of the boronate salt. Some suitable effervescing systems are described above under the heading “Oral Dosage Forms”. In some embodiments, the tablet is in monodose form; in others a single dosage will be obtained by combining a suitable number of tablets and usually combining all the tablets with a drinkable preparation, e.g. with an amount of from about 50 ml to about 150 ml water, or with such an amount of another beverage product. Suitable doses of boronic acid salt are as previously described in relation to powders and granules.

Where it is desired to include an acid in the effervescent tablet for the purpose of reducing the pH of the liquid to make it more pleasant to drink, this may be done by incorporating an excess of the organic acid used in the effervescent system, i.e. a single acid—e.g. citric acid—may serve as both a component of the effervescent system and a pH reductant for the liquid preparation made using the tablet.

Effervescent tablets and “fast melt” formulations may include as previously described, e.g. in most cases will contain an antimicrobial preservative and/or one or more flavour agents. One class of tablets and fast melt formulations comprises monodose tablets, or formulation units containing a single dose of boronic acid salt, as described above in relation to granules and powders. Alternatively, each tablet or formulation unit may contain a fraction of a dose, again as described above in relation to granules and powders.

Effervescent tablets will of course contain an effervescent system, as previously described.

The formulations may of course contain the active principle as a free boronic acid, or a salt or prodrug salt. Exemplary salts are not only base addition salts of alkali metals such as sodium, but also of alkaline earth metals, for example calcium. Further to be mentioned are base addition salts with organic bases, for example those disclosed earlier in this specification. Prodrugs have previously been described in the specification.

The disclosure is not restricted to reconstitutable formulations as described above. Thus certain aspects of the disclosure include or involve other oral formulations, for example tablets and capsules. Such aspects of the disclosure may include or involve parenteral, particularly intravenous formulations. Some of these aspects are:
the high purity salts
treatment (prevention) of flight DVT
treatment (prevention) of thrombosis in extracorporeal liver detoxification
use of compounds other than base addition salts to prevent thrombosis during haemodialysis.

In the case of CIHD or other intermittent apheresis, it is contemplated that intravenous TR 50c monososium salt would be administered to adult patients at a rate of from about 20 to 50 mg/hour which, using the conversion factor referred to previously, may readily be calculated as equivalent to approximately 0.035-0.089 millimoles TRI 50c per hour. Administration at a rate of about 0.035-0.089 millimoles TRI 50c per hour is therefore contemplated to be suitable for other TRI 50c salts and for salts of other boronic acids of similar potency (Ki of TRI 50c=7-22 nM) in the setting of CIHD and other intermittent apheresis procedures. Of course, the rate of administration may be adjusted in the clinical judgement of a medical practitioner, for example to take account of a patient's weight or other factors, and a rate of administration of about 0.025-0.125 mmoles/hour may therefore be mentioned. In the case of dissimilar potencies, say (Ki values outside the range of 5-25 nM), dosage rate may be adjusted accordingly. The activated clotting time (ACT) is a commonly used parameter for assessing the degree of anticoagulation and the target ACT in CIHD is 150 to 250 sec. The disclosure is not limited to administration in such quantities or regimens and includes dosages and regimens outside those described in this paragraph.

It is desirable in CIHD that as little water as possible be added during anticoagulation, since one of the purposes of CIHD is to remove water from the blood. It is therefore contemplated that a relatively soluble antithrombotic will be used, e.g. a sodium salt or a reaction product of a boronic acid and an aminosugar (for example N-methyl-D-glucamine) in the case of CIHD. Other procedures which involve intravenous anticoagulation may be less sensitive to the volume of water injected or infused and less soluble products may be preferred on a balance of factors. Thus, for example, it may in such instances be preferred to administer a salt of a divalent metal such as calcium or zinc for reason of stability. Magnesium is another pharmaceutically acceptable divalent metal. Trivalent metals may also be mentioned.

Examples of the procedures or settings less sensitive to volume of added water referred to in the previous paragraph include apheresis procedures other than haemodialysis, except in the setting of renal failure or disorder.

According to a further aspect, therefore, there is provided an oral or parenteral pharmaceutical formulation including a product as described herein, notably a product obtained or obtainable by the high purity process, in admixture with a pharmaceutically acceptable adjuvant, diluent or carrier.

Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules and tablets, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, the oral formulations may contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g., sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g., chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The presently disclosed product may be presented as solids in finely divided solid form, for example they may be micronised. Powders or finely divided solids may be encapsulated.

The active compound may be given as a single dose, in multiple doses or as a sustained release formulation.

In the case of tablets or capsules, the compounds, particularly the salts of amino- or peptido-boronic acids, may be administered in a form which prevents the salt from contact with the acidic gastric juice, such as enterically coated formulations, which thus prevent release of the salt until it reaches the duodenum.

The enteric coating is suitably made of carbohydrate polymers or polyvinyl polymers, for example. Examples of enteric coating materials include, but are not limited to, cellulose acetate phthalate, cellulose acetate succinate, cellulose hydrogen phthalate, cellulose acetate trimellitate, ethyl cellulose, hydroxypropyl-methylcellulose phthalate, hydroxypropylmethylcellulose acetate succinate, carboxymethyl ethylcellulose, starch acetate phthalate, amylose acetate phthalate, polyvinyl acetate phthalate, polyvinyl butyrate phthalate, styrene-maleic acid copolymer, methyl-acrylate-methacrylic acid copolymer (MPM-05), methylacrylate-methacrylic acid-methylmethacrylate copolymer (MPM-06), and methylmethacrylate-methacrylic acid co-polymer (Eudragit® L & S). Optionally, the enteric coating contains a plasticiser. Examples of the plasticiser include, but are not limited to, triethyl citrate, triacetin, and diethyl phthalate.

### Combination Therapy

The compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The salts of the disclosure may be combined and/or co-administered with any cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

The salts of the disclosure may be combined and/or co-administered with any antithrombotic agent with a different mechanism of action, such as the antiplatelet agents acetylsalicylic acid, ticlopidine, clopidogrel, thromboxane receptor and/or synthetase inhibitors, prostacyclin mimetics and phosphodiesterase inhibitors and ADP-receptor (P₂ T) antagonists.

The products of the disclosure may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

The salts of the disclosure may be combined and/or co-administered with a cardioprotectant, for example an adenosine A1 or A3 receptor agonist.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

The formulations and dosage forms of the disclosure include those in which the salt is an alkali metal salt, for example a lithium, sodium or potassium salt, of which sodium salts may be mentioned as particular salts. Another class of formulations contains aminosugar salts of the disclosed boronic acids, for example N-methyl-D-glucamine salts. The salts mentioned in this paragraph may be administered as solutions in water, typically containing one or more additives, for example isotonicity agent(s) and/or antioxidant(s). A suitable way to store the salts is in solid form, for example as dry powder, and to make them up into solutions for administration prior to administration.

It will be understood from the aforegoing that there are provided pharmaceutical products comprising a mono alkali metal or hemi alkaline earth metal salt of a boronic acid of Formula (I) in the form of a product suitable for reconstitution into an aqueous read-to-drink formulation. The product suitable for reconstitution may comprise a sachet containing powder or granules or it may comprise an effervescent tablet. One example is a monosodium salt of a boronic acid of Formula (I), particularly TRI 50c, in dry powder or granular form for reconstitution as a drinkable liquid formulation, particularly a solution. Another example is a hemicalcium salt of a boronic acid of Formula (I), particularly TRI 50c, in dry powder or granular form for reconstitution as a drinkable liquid formulation, particularly a solution. The salt may be a lyophilisate. The dry powder or granular material may be packaged in a sachet. As an alternative to such dry powder or granular material, the monosodium or hemicalcium salt may be in the form of an effervescent tablet.

### EXAMPLES

### Examples 1 to 4

### Introductory Remarks

### Apparatus

Throughout the following procedures of Examples 1 to 4, standard laboratory glassware and, where appropriate, specialised apparatus for handling and transferring of air sensitive reagents are used.

All glassware is heated at 140-160° C. for at least 4 hours before use and then cooled either in a desiccator or by assembling hot and purging with a stream of dry nitrogen.

### Solvents

The organic solvents used in the procedures of Examples 1 to 4 are all dry. Suitably, they are dried over sodium wire before use.

### Dryness

In the drying procedures of Example 1 to 4, products are tested for dryness (including dryness in terms of organic solvent) by observing weight loss on drying. The following procedure was followed to determine loss on drying: a sample was placed in a vacuum drier and dried at 40° C. at 100 mbar for 2 hours. Products are considered dry when the decrease in weight upon drying is less than 0.5% of the total weight of the starting material.

Examples 1 to 4 describe performance of the following reaction scheme and conversion of the resultant TRI 50c to sodium and calcium salts thereof:

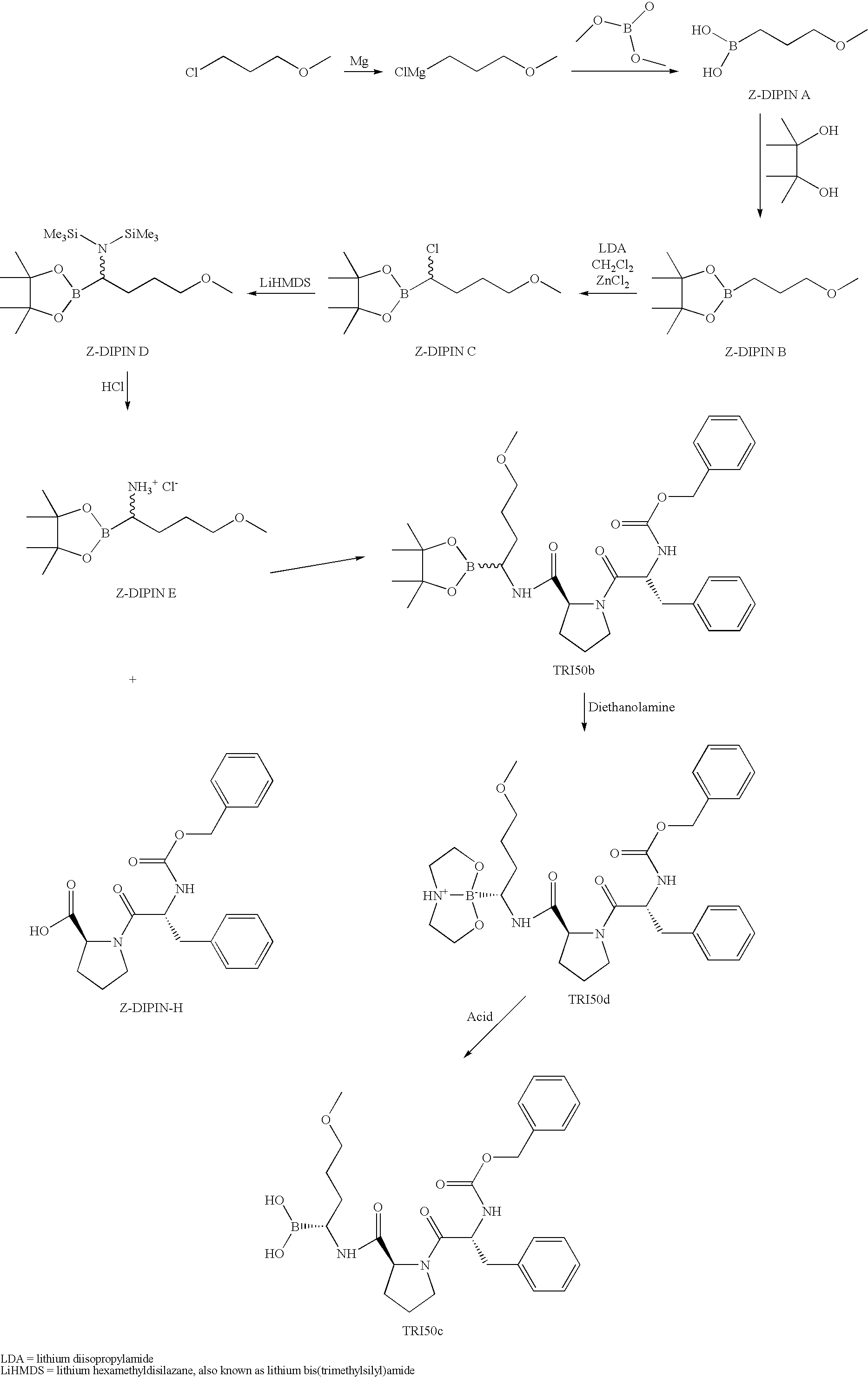

### Example 1

### Synthesis of TRI 50B

### Step 1: Z-DIPIN B

### Procedure A

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux until all the magnesium is consumed. After the reaction is finished, the reaction mixture is cooled to ambient temperature and slowly added to a solution of 64.4 g (620 mmole) trimethylborate in 95 ml dry tetrahydrofuran; the latter solution is cooled to below 0° C. and, if it warms up during the course of the reaction, the reaction mixture must be added to it sufficiently slowly to maintain the temperature of this solution below 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The layers are allowed to settle and the phases are separated. The aqueous layer is rewashed three times with 200 ml tert.-butylmethylether. The combined organic layers are allowed to settle and additional water separated from this solution is removed. The organic layer is dried over magnesium sulfate, filtered and evaporated to dryness. The evaporation residue is filtered from the precipitated solid and the filtrate dissolved in 175 ml toluene. 34.8 g (292 mmole) pinacol is charged to the solution followed by stirring at ambient temperature for not less than 10 hours. The solution is evaporated to dryness, dissolved in 475 ml n-heptane and washed three times with 290 ml saturated aqueous solution of sodium hydrogen carbonate. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70%) Z-DIPIN B (oil)

### Procedure B

17.8 g (732.5 mmole) magnesium turnings, 0.1 g (0.4 mmole) iodine and 127 ml dry tetrahydrofuran are charged and heated to reflux. Then 15 ml of a solution of 66 g (608 mmole) 1-chloro-3-methoxypropane in 185 ml dry tetrahydrofuran are added and stirred under reflux until the vigorous reaction starts. After the initial exotherm ceases, the solution of 1-chloro-3-methoxypropane is added slowly to maintain gentle reflux. After the reaction is finished, the reaction mixture is cooled to ambient temperature and slowly added to a solution of 64.4 g (620 mmole) trimethylborate in 95 ml dry tetrahydrofuran, maintaining the temperature of this solution below minus 65° C. Upon complete addition, the reaction mixture is allowed to warm to about 0° C. and stirred for another 60 minutes. Then a solution of 22.4 ml sulfuric acid in 400 ml water is added slowly so as to maintain the temperature below 20° C. The organic solvent is removed by distillation under vacuum. 300 ml n-heptane is charged to the aqueous solution of the evaporation residue followed by addition of 34.8 g (292 mmole) pinacol. The two-phase-mixture is stirred at ambient temperature for not less than 2 hours. After allowing the layers to settle, the aqueous phase is separated. 300 ml n-heptane is charged to the aqueous solution and the two-phase-mixture is stirred at ambient temperature for not less than 2 hours. After allowing the layers to settle, the aqueous phase is separated. The organic layers are combined and washed once with 200 ml water, followed by 200 ml saturated sodium hydrogen carbonate solution and two further washes with 200 ml water each. The n-heptane solution is evaporated to dryness and the evaporation residue distilled and the fraction with Bp 40-50° C. at 0.1-0.5 mbar recovered.

Boiling point: 40-50° C./0.1-0.5 mbar

Yield: 40.9 g (70-85%) Z-DIPIN B (oil)

### Step 2: Z-DIPIN C

16.6 g (164 mmole) diisopropylamine and 220 ml tetrahydrofuran are charged and cooled to −30 to −40° C. To this solution 41.8 g (163 mmole) n-butyl lithium, 25% in n-heptane is added, followed by stirring at 0 to −5° C. for one hour. This freshly prepared solution of lithium diisopropylamide is cooled to −30° C. and then added to a solution of 27.9 g (139 mmole) Z-DIPIN B in 120 ml tetrahydrofuran and 35.5 g (418 mmole) dichloromethane at a temperature between 60 and −75° C. The solution is stirred at that temperature for half an hour followed by addition of 480 ml (240 mmole) 0.5N anhydrous Zinc(II)-chloride in tetrahydrofuran or 32.5 g (240 mmole) anhydrous solid Zinc(II)-chloride. After stirring at −65° C. for one hour, the reaction mixture is allowed to warm to ambient temperature and stirred for another 16-18 hours. The reaction mixture is evaporated to dryness (i.e. until solvent is removed) and followed by addition of 385 ml n-heptane. The reaction mixture is washed with 150 ml 5% sulfuric acid, with 190 ml saturated sodium hydrogen carbonate solution, and 180 ml saturated sodium chloride solution. The organic layer is dried over magnesium sulfate, filtered and evaporated to dryness (i.e. until solvent is removed). The oily residue is transferred into the next step without further purification.

Yield: 19 g (55%) Z-DIPIN C

### Step 3: Z-DIPIN D

To a solution of 23.8 g (148 mmole) hexamethyldisilazane in 400 ml tetrahydrofuran at −15° C. is added 34.7 g (136 mmole) n-butyl lithium, 25% in n-heptane and stirred for one hour. The solution is cooled to −55° C. followed by the addition of 30.6 g (123 mmole) Z-DIPIN C dissolved in 290 ml tetrahydrofuran and 35 ml tetrahydrofuran to this freshly prepared solution of LiHMDS. The solution is allowed to warm to ambient temperature and stirred for 12 hours. The reaction mixture is evaporated to dryness, the evaporation residue dissolved in 174 ml n-heptane, washed with 170 ml water and 75 ml saturated sodium chloride solution. The organic phase is dried over magnesium sulfate, filtered and evaporated to complete dryness (i.e. until solvent is removed). The oily residue is dissolved in 100 g n-heptane. This solution is carried over into the next step without further purification.

Yield: 32.2 g (70%) Z-DIPIN D

Step 4: Z-DIPIN (TRI50b, crude)

A solution of 26.6 g (71 mmole) Z-DIPIN D in 82.6 g n-heptane is diluted with 60 ml n-heptane and cooled to −60° C. followed by introduction of 10.5 g (285 mmole) hydrogen chloride. The reaction mixture is subsequently evacuated and flushed with nitrogen, while the temperature is increased in increments of about 20° C. to ambient temperature. The solvent is removed from the oily precipitate and replaced several times by 60 ml fresh n-heptane. The oily residue is dissolved in 60 ml tetrahydrofuran (Solution A).

To a different flask 130 ml tetrahydrofuran, 24.5 g (61.5 mmole) Z-D-Phe-Pro-OH and 6.22 g (61.5 mmole) N-methylmorpholine are charged and cooled to −20° C. To this solution a solution of 8.4 g (61.5 mmole) isobutylchloroformate in 20 ml tetrahydrofuran is added and stirred for 30 minutes, followed by addition of Solution A at −25° C. Upon complete addition, up to 16 ml (115 mmole) triethylamine is added to adjust the pH to 9-10, measured using a pH stick. The reaction mixture is allowed to warm to ambient temperature and stirred for 3 hours, still under nitrogen. The solvent is evaporated to dryness and the evaporation residue dissolved in 340 ml tert.-butylmethylether (t-BME). The solution of Z-DIPIN in t-BME is washed twice with 175 ml 1.5% hydrochloric acid. The combined acidic washes are given a rewash with 175 ml t-BME. The combined organic layers are washed with 175 ml water, with 175 ml saturated sodium hydrogen carbonate solution, with 175 ml 25% sodium chloride solution, dried over magnesium sulfate and filtered. This solution is carried over into the next step without further purification.

Yield: 29.9 g (80%) Z-DIPIN

### Example 2

### Synthesis of TRI 50D (Diethanolamine Adduct of TRI 50C)

The starting material used in this Example is the solution of TRI 50b (“Z-DIPIN”) obtained in Example 1. The solution is carried forward to the synthesis of TRI 50d without further purification. The solution of Z-DIPIN in t-BME (containing 7.0 g (11.5 mmole) (R,S,R) TRI 50b, calculated based on HPLC results of Z-DIPIN) is evaporated to dryness and the evaporation residue dissolved in 80 ml diethylether. 1.51 g (14.4 mmole) diethanolamine is added and the mixture heated at reflux for at least 10 hours, during which process the product precipitates. The suspension is cooled to 5-10° C., filtered and the filter residue washed with diethylether.

To improve chiral and chemical purity the wet filter cake (7 g) is dissolved in 7 ml dichloromethane, cooled to 0-5° C. and the product precipitated by addition of 42 ml diethylether and filtered. The isolated wet product is dried at 35° C. in vacuum or at least 4 hours, until day.

Yield: 5.5 g (80%) Tri50d

Melting Point: 140-145° C.

### Example 3

### Preparation of Sodium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. A precipitate forms in the organic phase. After stirring, the layers are allowed to settle and separated. The aqueous layer is rewashed twice with 2.2 L dichloromethane. The combined organic layers are washed with a solution of 625 g ammonium chloride in 2.25 L water. (The ammonium chloride buffers the pH of the aqueous extractions to be within a range of from about pH 1-2 to about pH 4-5, as strongly acidic conditions might cleave peptide bonds). The organic phase is dried over magnesium sulfate, filtered and the filtrate evaporated to dryness. An assay of the free boronic acid is performed (by the RP HPLC method of Example 38 for at most 30 mins (optionally about 20 min) at room temperature) and the amounts of the solvents and base for conversion of the acid to the salt are calculated. If 2.5 mol of the free acid is obtained, the evaporation residue is dissolved in 5 L acetonitrile followed by addition of a solution of 100 g (2.5 mole) sodium hydroxide as a 5% solution in 2.2 L water. The solution is stirred for two hours at ambient temperature (e.g. 15-30° C., optimally room temperature) and then evaporated in vacuum (of ca. 10 mmHg) at a temperature not exceeding 35° C. The evaporation residue is repeatedly dissolved in 3.5 L fresh acetonitrile and evaporated to dryness to remove traces of water. If the evaporation residue is dry, it is dissolved in 3 L acetonitrile (or alternatively in 6 L THF) and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum at a temperature initially of about 10° C. and then increasing to a limit of about 35° C., until dry.

Yield: 1.0 kg (70%) Tri50c sodium salt.

### Example 4

### Preparation of Calcium Salt of TRI50C

1.5 kg (2.5 mole) TRI50d from Example 2 is dissolved in 10.5 L dichloromethane. 11 L 2% hydrochloric acid is added and the mixture is stirred for at most 30 minutes (optimally about 20 minutes) at room temperature. After stirring the layers are allowed to settle and separated. The aqueous layer is given a rewashed twice with 2.2 L dichloromethane. The combined organic layers are washed with a solution of 625 g ammonium chloride in 2.25 L water. The organic phase is dried over magnesium sulfate, filtered and the filtrate evaporated to dryness. An assay of the free boronic acid is performed and the amounts of the solvents and base for conversion of the acid to the salt are calculated. If 2.5 mol of the free acid is obtained, the evaporation residue is dissolved in 5 L acetonitrile followed by addition of a suspension of 93 g (1.25 mole) calcium hydroxide in 1 L water. The solution is stirred for two hours at ambient temperature (e.g. 15-30° C., optimally room temperature) and then evaporated under vacuum (of ca. 10 mmHg) at a temperature initially of about 10° C. and then increasing to a limit of about 35° C. The evaporation residue is repeatedly dissolved in 3.5 L fresh acetonitrile and evaporated to dryness to remove traces of water. If the evaporation residue is dry, it is dissolved in 6 L tetrahydrofuran and slowly added to a mixture of 32 L n-heptane and 32 L diethylether. The addition is performed slowly enough to avoid lumping or sticking of the product and is carried out over a period of not less than 30 minutes. The precipitated product is filtered off, washed with n-heptane and dried under vacuum (of ca. 10 mmHg) at a temperature below 35° C. until dry.

Yield: 0.98 kg (70%) Tri50c calcium salt.

The procedures of Examples 1 to 4 may be scaled up and, if operated carefully, will produce highly pure salts. In the diethanolamine precipitation step it is important to use 1.25 equivalents of diethanolamine per equivalent of (R,S,R) TRI 50b. In the hydrolysis of the diethanolamine ester, it is important to avoid excessively long contact with the aqueous acid. Likewise the TRI 50b should be synthesised via the Grignard reaction to Z-DIPIN A.

### Example 5

### Alternative Conversion of TRI 50B to TRI 50C

The synthetic procedures described in this and subsequent synthetic examples were generally performed under nitrogen and using dry solvents as supplied from commercial sources.

1. Approximately 300 g of TRI 50b, obtained by the HPLC purification of racemic TRI 50b) were dissolved in approximately 2.5 L diethylether. It is estimated that different batches of TRI 50b had isomeric purities ranging from 85% R,S,R to in excess of 95% R,S,R.
2. Approximately 54 ml diethanolamine were added (1:1 stoichiometry with total TRI 50b content), and the mixture was refluxed at 40° C.
3. The precipitated product was removed, washed several times with diethylether and dried.
4. The dry product was dissolved in CHCl₃. Hydrochloric acid (pH 1) was added and the mixture was stirred approximately 1 h at room temperature.
5. The organic layer was removed and washed with NH₄Cl solution.
6. The organic solvent was distilled off and the residual solid product was dried.
Typical yield: Approximately 230 g

### Example 6

### Preparation of Lithium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added LiOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water necessary with light warming for about 20 minutes. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield 17.89 g.

### Microanalysis:

| C % Found | H % Found | N % Found | B % Found | Metal % Found |
|---|---|---|---|---|
| (Calc.) | (Calc.) | (Calc.) | (Calc.) | (Calc.) |
| 57.14 | 6.60 | 7.34 | 2.07 | Li 1.26 |
| (61.03) | (6.64) | (7.90) | (2.03) | (1.31) |

### Example 7

### UV/Visible Spectra of Lithium Salt of TRI50C

UV/Visible spectra of the salt resulting from the procedure of Example 6 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 451

### Example 8

### Aqueous Solubility of Lithium Salt of TRI50C

The salt used in this Example was made using a modification of the process described in Example 6. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The lithium salt was comparatively soluble and so was redissolved at 50 mg/ml in the same manner previously described.

Solubility when dissolved at 25 mg/ml: 43 mM (23 mg/ml).

Solubility when dissolved at 50 mg/ml: 81 mM (43 mg/ml).

### Example 9

### Preparation of Sodium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added NaOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 15-20 minutes. The solution is filtered through filter paper and evacuated to dryness, again with the temperature of the solution not exceeding 37° C. The resultant product is dried under vacuum overnight to normally yield a white brittle solid. The product may be present as an oil or tacky solid due to residual water, in which case it is dissolved in ethyl acetate and evacuated to dryness to produce the product as a white solid.

The salt was then dried under vacuum over silica to constant weight (72 h).

Yield: Over 50%.

### Microanalysis:

| C % Found | H % Found | N % Found | B % Found | Metal % Found |
|---|---|---|---|---|
| (Calc.) | (Calc.) | (Calc.) | (Calc.) | (Calc.) |
| 59.93 | 6.47 | 7.31 | 1.91 | Na 3.81 |
| (59.24) | (6.44) | (7.67) | (1.98) | (4.20) |

### Example 10

### UV/Visible Spectra of Sodium Salt of TRI50C

UV/Visible spectra of the sodium salt resulting from the procedure of Example 9 were recorded in distilled water at 20° C. from 190 nm to 400 nm. The salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 415.

### Example 11

### Aqueous Solubility of Sodium Salt of TRI50C

The salt used in this Example was made using a modification of the process described in Example 9. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material. The sodium salt was comparatively soluble and so was redissolved at 50 mg/ml in the same manner previously described.

Solubility when dissolved at 25 mg/ml: 44 mM (25 mg/ml).

Solubility when dissolved at 50 mg/ml: 90 mM (50 mg/ml).

### Example 12

### Preparation of Potassium Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added KOH as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 1 L distilled water with warming to 37° C. for about 2 hours. The solution is filtered through filter paper and evacuated to dryness, again with the temperature of the solution not exceeding 37° C. The resultant product is dried under vacuum overnight to normally yield a white brittle solid.

Yield: 14.45 mg.

The salt was then dried under vacuum over silica to constant weight (72 h).

### Microanalysis:

| C % Found | H % Found | N % Found | B % Found | Metal % Found |
|---|---|---|---|---|
| (Calc.) | (Calc.) | (Calc.) | (Calc.) | (Calc.) |
| 54.84 | 6.25 | 7.02 | 2.01 | K 4.29 |
| (57.55) | (6.26) | (7.45) | (1.92) | (6.94) |

### Example 13

### UV/Visible Spectra of Potassium Salt of TRI50C

UV/Visible spectra of the potassium salt resulting from the procedure of Example 12 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 438.

### Example 14

### Aqueous Solubility of Potassium Salt of TRI50C

The salt used in this Example was made using a modification of the process described in Example 12. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 29 mM (16 mg/ml).

### Example 15

### Preparation of Zinc Salt of TRI 50C

The relative solubility of zinc hydroxide is such that, if the hydroxide had been used to prepare the corresponding TRI 50c salt using the procedure of Example 6, they would not have resulted in homogeneous salt formation. A new method was therefore developed to prepare the zinc salt, as described in this and the next examples.

TRI 50c sodium salt (2.24 g, 4.10 mM) was dissolved in distilled water (100 ml) at room temperature and zinc chloride in THF (4.27 ml, 0.5M) was carefully added with stirring. A white precipitate that immediately formed was filtered off and washed with distilled water. This solid was dissolved in ethyl acetate and washed with distilled water (2×50 ml). The organic solution was evacuated to dryness and the white solid produced dried over silica in a desiccator for 3 days before microanalysis. Yield 1.20 g.

¹H NMR 400 MHz, δ_{H}(CD₃OD) 7.23-7.33 (20H, m, ArH), 5.14 (4H, m, PhCH₂O), 4.52 (4H, m, αCH), 3.65 (2H, m), 3.31 (12H, m), 3.23 (6H, s, OCH₃), 2.96 (4H, d, J 7.8 Hz), 2.78 (2H, m), 2.58 (2H, m), 1.86 (6H, m), 1.40 (10H, m).

¹³C NMR 75 MHz δ_{C}(CD₃OD) 178.50, 159.00, 138.05, 137.66, 130.54, 129.62, 129.50, 129.07, 128.79, 128.22, 73.90, 67.90, 58.64, 58.18, 56.02, 38.81, 30.06, 28.57, 28.36, 25.29.

FTIR (KBr disc) νₘₐₓ (cm⁻¹) 3291.1, 3062.7, 3031.1, 2932.9, 2875.7, 2346.0, 1956.2, 1711.8, 1647.6, 1536.0, 1498.2, 1452.1, 1392.4, 1343.1, 1253.8, 1116.8, 1084.3, 1027.7, 916.0, 887.6, 748.6, 699.4, 595.5, 506.5.

### Example 16

### Preparation of Arginine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added arginine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 2 L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid.

The salt was then dried under vacuum over silica to constant weight (72 h).

Yield: 10.54 g.

### Microanalysis:

| C % Found | H % Found | N % Found | B % Found |
|---|---|---|---|
| (Calc.) | (Calc.) | (Calc.) | (Calc.) |
| 52.47 | 7.12 | 15.25 | 1.52 |
| (56.65) | (7.20) | (14.01) | (1.54) |

### Example 17

### UV/Visible Spectra of Arginine Salt of TRI50C

UV/Visible spectra of the salt resulting from the procedure of Example 15 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 406.

### Example 18

### Aqueous Solubility of Arginine Salt of TRI50C

The salt used in this Example was made using a modification of the process described in Example 16. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 14 mM (10 mg/ml).

### Example 19

### Preparation of Lysine Salt of TRI50C

Cbz-Phe-Pro-BoroMpg-OH obtained by the method of Example 5 (20.00 g, 38.1 mM) is dissolved in acetonitrile (200 ml) with stirring at room temperature. To this solution is added L-lysine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 3 L distilled water with warming to 37° C. for 2 hours. The solution is filtered through filter paper and evacuated to dryness, again with the temperature of the solution not exceeding 37° C. The resultant product is dried under vacuum overnight to normally yield a white brittle solid. The product may be present as an oil or tacky solid (due to residual water), in which case it is then dissolved in ethyl acetate and evacuated to dryness to produce the product as a white solid.

The salt was then dried under vacuum over silica to constant weight (72 h).

Yield: 17.89.

### Microanalysis:

| C % Found | H % Found | N % Found | B % Found |
|---|---|---|---|
| (Calc.) | (Calc.) | (Calc.) | (Calc.) |
| 57.03 | 7.43 | 10.50 | 1.72 |
| (59.11) | (7.36) | (10.44) | (1.61) |

### Example 20

### UV/Visible Spectra of Lysine Salt of TRI50C

UV/Visible spectra of the salt resulting from the procedure of Example 19 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 437.

### Example 21

### Aqueous Solubility of Lysine Salt of TRI50C

The salt used in this Example was made using a modification of the process described in Example 19. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 13 mM (8.6 mg/ml).

### Example 22

### PREPARATION of N-METHYL-D-GLUCAMINE SALT OF TRI50C

Cbz-Phe-Pro-BoroMpg-OH obtained by the method of Example 5 (20.00 g, 38.1 mM) is dissolved in acetonitrile (200 ml) with stirring at room temperature. To this solution is added N-methyl-D-glucamine as a 0.2M solution in distilled water (190 ml). The resultant clear solution is stirred for 2 hours at room temperature and then evacuated to dryness under vacuum with its temperature not exceeding 37° C. The resultant oil/tacky liquid is redissolved in 500 ml distilled water with light warming for about 20 minutes. The solution is filtered through filer paper and evacuated to dryness, again with the temperature of the solution not exceeding 37° C., or freeze dried. The resultant product is dried under vacuum overnight to normally yield a white brittle solid.

The salt was then dried under vacuum over silica to constant weight (72 h).

Yield: 21.31 g.

### Microanalysis:

| C % Found | H % Found | N % Found | B % Found |
|---|---|---|---|
| (Calc.) | (Calc.) | (Calc.) | (Calc.) |
| 56.67 | 7.28 | 7.74 | 1.63 |
| (56.67) | (7.41) | (7.77) | (1.50) |

### Example 23

### UV/VISIBLE SPECTRA of N-METHYL-D-GLUCAMINE SALT of TRI50C

UV/Visible spectra of the salt resulting from the procedure of Example 22 were recorded in distilled water at 20° C. from 190 nm to 400 nm. TRI50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 433.

### Example 24

### AQUEOUS SOLUBILITY OF N-METHYL-D-GLUCAMINE SALT OF TRI50C

The salt used in this Example was made using a modification of the process described in Example 22. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt was observed to fully dissolve. The salt was comparatively soluble and so was redissolved at 50 mg/ml in the same manner previously described.

Solubility when dissolved at 25 mg/ml: 35 mM (25 mg/ml).

Solubility when dissolved at 50 mg/ml: 70 mM (50 mg/ml).

### Example 2

### Alternative Preparation of Arginine Salt of TRI50C

The arginine salt is formed simply by adding a slight molar excess of L-arginine to a solution of 0.2-0.3 mmol of TRI 50c in 10 ml of ethyl acetate. The solvent is evaporated after one hour, and the residue is triturated twice with hexane to remove excess arginine.

### Example 26

### First Preparation of Calcium Salt of TRI 50C

Cbz-Phe-Pro-BoroMpg-OH (20.00 g, 38.1 mM) obtained by the method of Example 5 is dissolved in acetonitrile (200 ml) with stirring at room temperature. To this solution is added Ca(OH)₂ as a 0.1M solution in distilled water (190 ml). The resultant clear solution is stirred for 2 hours at room temperature and then evacuated to dryness under vacuum with its temperature not exceeding 37° C. The resultant product is a white brittle solid.

The salt was then dried under vacuum over silica to constant weight (72 h).

Yield: 17.69 g.

### Example 27

### Second Alternative Preparation of Calcium Salt of TRI 50C

50.0 g TRI 50c (95.2 mmol) were dissolved under stirring in 250 ml acetonitrile at room temperature and then cooled with an ice bath. To this ice cooled solution 100 ml of an aqueous suspension of 3.5 g (47.6 mmol) calcium hydroxide was added dropwise, stirred for 2.5 hours at room temperature, filtered and the resulting mixture evaporated to dryness, the temperature not exceeding 35° C. The clear yellowish oily residue was redissolved in 200 ml acetone and evaporated to dryness. The procedure of redissolving in acetone was repeated one more time to obtain colourless foam.

This foam was redissolved in 100 ml acetone, filtered and added dropwise to an ice cooled solution of 1100 ml petrol ether 40/60 and 1100 ml diethylether. The resulting colourless precipitate was filtered, washed two times with petrol ether 40/60 and dried under high vacuum, yielding 49.48 g of a colourless solid (92%), with a purity of 99.4% according to an HPLC measurement.

### Example 28

### UV/Visible Spectra of Calcium Salt of TRI 50C

UV/Visible spectra of the salt resulting from the procedure of Example 26 were recorded in distilled water at 20° C. from 190 nm to 400 nm. TRI 50C and the salt gave λₘₐₓ at 210 and 258 nm. The weight of the dried salt was then measured for the purposes of calculating the extinction coefficient. The λₘₐₓ at 258 nm was used. The extinction coefficient was calculated using the formula:—

A=εcI where A is the absorbance
C is the concentration
I the path length of the UV cell
and ε is the extinction coefficient.
   Extinction coefficient: 955.

### Example 29

### Aqueous Solubility of Calcium Salt of TRI 50C

The salt used in this Example was made using a modification of the process described in Example 27. The modified process differs from that described in that 100 mg of TRI 50c was used as starting material, the product of the redissolution in water was dried by freeze drying and the filtration was carried out through a 0.2 μm filter. The salt is believed to contain about 85% of R,S,R isomer.

To determine maximum aqueous solubility 25 mg of the dried salt were shaken in water at 37° C., the sample filtered and the UV spectrum measured. The salt left a white residue of undissolved material.

Solubility when dissolved at 25 mg/ml: 5 mM (5 mg/ml).

### Example 30

### In Vitro Activity of Calcium Salt of TRI 50C

TRI 50c calcium salt was assayed as an inhibitor of human α-thrombin by an amidolytic assay (J. Deadman et al, J. Med. Chem. 38:15111-1522, 1995, which reports a Ki value of 7 nM for TRI 50b).

The inhibition of human α-thrombin therefore, was determined by the inhibition of the enzyme catalysed hydrolysis of three different concentrations of the chromogenic substrate S-2238.

200 μl of sample or buffer and 50 μl of S-2238 were incubated at 37° C. for 1 minute and 50 μl of human α-thrombin (0.25 NIHμ/ml) was added. The initial rate of inhibited and uninhibited reactions were recorded at 4.5 nm. The increase in optical density was plotted according to the method of Lineweaver and Burke. The Km and apparent Km were determined and Ki was calculated using the relationship.

$$V = \frac{V max}{1 + \frac{Km}{[ S ]} \cdot ( 1 + \frac{[ I ]}{Ki} )}$$

The buffer used contained 0.1M sodium phosphate, 0.2M NaCl, 0.5% PEG and 0.02% sodium azide, adjusted to pH 7.5 with orthophosphoric acid.

The samples consist of the compound dissolved in DMSO.

The reader is referred to Dixon, M and Webb, E. C., “Enzymes”, third edition, 1979, Academic Press, the disclosure of which is incorporated herein by reference, for a further description of the measurement of Ki.

TRI 50c calcium salt was observed to have a Ki of 10 nM.

### Example 31

### Preparation of Magnesium Salt of TRI 50C

TRI 50c (1.00 g, 1.90 mM) was dissolved in methanol (10 ml) and stirred at room temperature. To this solution was added magnesium methoxide (Mg(CH₃O)₂) in methanol (1.05 ml, 7.84 wt %). This solution was stirred for 2 hours at room temperature filtered and evacuated to 5 ml. Water (25 ml) was then added and the solution evacuated down to dryness to yield a white solid. This was dried over silica for 72 hours before being sent for microanalysis. Yield 760 mg.

¹H NMR 300 MHz, δ_{H}(CD₃C(O)CD₃) 7.14-7.22 (20H, m), 6.90 (2H, m), 4.89 (4H, m, PhCH₂O), 4.38 (2H, m), 3.40 (2H, br s), 2.73-3.17 (20H, broad unresolved multiplets), 1.05-2.10 (16H, broad unresolved multiplets).

¹³C NMR 75 MHz δ_{C}(CD₃C(O)CD₃) 206.56, 138.30, 130.76, 129.64, 129.31, 129.19, 129.09, 128.20, 128.04, 74.23, 73.55, 67.78, 58.76, 56.37, 56.03, 48.38, 47.87, 39.00, 25.42, 25.29.

FTIR (KBr disc) νₘₐₓ (cm⁻¹) 3331.3, 3031.4, 2935.3, 2876.9, 2341.9, 1956.1, 1711.6, 1639.9, 1534.3, 1498.1, 1453.0, 1255.3, 1115.3, 1084.6, 1027.6, 917.3, 748.9, 699.6, 594.9, 504.5, 467.8.

### Example 32

### Solubility of TRI 50C

The UV/visible spectra of TRI50c resulting from the procedure of Example 5 and its solubility were obtained as described above in relation to the salts. The solubility of TRI50c when dissolved at 50 mg/ml was 8 mM (4 mg/ml).

### Example 33

### Analysis Of Sodium, Calcium, Magnesium and Zinc Salts of (R,S,R) TRI 50C

The following salts were prepared using a boronate:metal stoichiometry of n:1, where n is the valency of the metal, using (R,S,R) TRI 50c of higher chiral purity than that used to prepare the salts described in Examples 8, 11, 14, 18, 21, 24 and 29.

### A. Sodium Salt Product of Example 9)

Analytical data

HPLC or LC/MS: HPLC betabasic C18 Column,
CH₃CN, Water
Estimated Purity: >95% by UV (λ₂₁₅ₙₘ)

Micro analysis:

| | | Calcd. | Found. |
|---|---|---|---|
| C: | | 59.24 | 59.93 |
| H: | | 6.44 | 6.47 |
| N: | | 7.67 | 7.31 |
| Other: | B: | 1.98 | 1.91 |
| | Na: | 4.20 | 3.81 |

Physical Properties

Form: Amorphous solid
Colour: White
Melting Point: N/A
Solubility: Soluble in aqueous media
ca~50 mg/ml
M_{w}: 547.40

### B. Calcium Salt (Product of Example 26)

Analytical data

HPLC or LC/MS: HPLC betabasic C18 Column,
CH₃CN, Water
Estimated Purity: >95% by UV (λ₂₁₅ₙₘ)

Micro analysis:

| | | Calcd. | Found. |
|---|---|---|---|
| C: | | 59.27 | 55.08 |
| H: | | 6.48 | 6.43 |
| N: | | 7.71 | 7.08 |
| Other: | B: | 1.99 | 2.01 |
| | Ca: | 3.68 | 3.65 |

Physical Properties

Form: Amorphous solid
Colour: White
Melting Point: N/A
Solubility: Soluble in aqueous media
ca~4 mg/ml
M_{w}: 1088.89

### C. Magnesium Salt (Product of Example 31)

Analytical data

HPLC or LC/MS: HPLC betabasic C18 Column,
CH₃CN, Water
Estimated Purity: >90% by UV (λ₂₁₅ₙₘ)

Micro analysis:

| | | Calcd. | Found. |
|---|---|---|---|
| C: | | 60.44 | 57.25 |
| H: | | 6.57 | 6.71 |
| N: | | 7.83 | 7.45 |
| Other: | B: | 2.01 | 2.02 |
| | Mg: | 2.26 | 2.12 |

Physical Properties

Form: Amorphous solid
Colour: White
Melting Point: N/A
Solubility: Soluble in aqueous media
ca~7 mg/ml
M_{w}: 1073.12

### D. Zinc Salt Product of Example 15)

Analytical data

HPLC or LC/MS: HPLC betabasic C18 Column,
CH₃CN, Water
Estimated Purity: >95% by UV (λ₂₁₅ₙₘ)

Micro analysis:

| | | Calcd. | Found. |
|---|---|---|---|
| C: | | 58.21 | 56.20 |
| H: | | 6.33 | 6.33 |
| N: | | 7.54 | 7.18 |
| Other: | B: | 1.94 | 1.84 |
| | Zn: | 5.87 | 7.26 |

Physical Properties

Form: Amorphous solid
Colour: White
Melting Point: N/A
Solubility: Soluble in aqueous media
ca~2 mg/ml
M_{w}: 1114.18

Notes: The trigonal formula of the acid boronate is used in the calculated microanalyses. It is believed that a lower sodium salt solubility is reported in example 11 because the salt tested in example 11 had lower chiral purity.

### CONCLUSION

The zinc, calcium and magnesium salts have all been prepared with a stoichiometry of one metal ion to two molecules of TRI 50c. The values found for the calcium and magnesium salts are close to and thus consistent with those calculated for this 1:2 stoichiometry. For the zinc salt an excess of zinc was found; nonetheless, the zinc salt comprises a significant proportion of acid boronate. The sodium salt has been prepared with a stoichiometry of one metal ion to one molecule of TRI 50c.

The value found for the sodium salt is close to and thus consistent with that calculated for this 1:1 stoichiometry.

### Example 34

### Stability

An assay of TRI 50c and its sodium and lysine salts before and after drying.

### 1. Tabulated Results

TABLE 1

| Compound | Amount [μg/mL] | Purity (% area) |
|---|---|---|
| TRI 50c dry | 1000.0 | 82.00 |
| TRI 50c non-dried | 947.3 | 85.54 |
| TRI 50c Na salt dry | 1024 | 98.81 |
| TRI 50c Na salt non-dried | 1005.8 | 98.61 |
| TRI 50c Lys salt dry | 813.3 | 90.17 |
| TRI 50c Lys salt non-dried | 809.8 | 92.25 |

The purity of the acid was lowered by the drying process but the purity of the salts was less affected; the purity of the sodium salt was not significantly reduced. Large differences in response factors will reduce the actual impurity levels, however.

### 2. Analytical Procedure

### 2.1 Sample Preparation

TRI 50c and its Na, Li and Lys salts were weighed into HPLC vials and stored in a desiccator over phosphorus pentoxide for 1 week. For sample analysis, 5 mg of dried and non-dried material was weighed in a 5 mL volumetric flask and dissolved in 1 mL acetonitrile and filled up with demineralised water to 5 mL.

### 3. Data Evaluation

The quantitative evaluation was performed using an HPLC-PDA method.

### 4. Analytical Parameters

### 4.1 Equipment and Software

| | |
|---|---|
| Autosampler | Waters Alliance 2795 |
| Pump | Waters Alliance 2795 |
| Column oven | Waters Alliance 2795 |
| Detection | Waters 996 diode array, MS-ZQ 2000 single quad |
| Software version | Waters Millennium Release 4.0 |

### 4.2 Stationary Phase

| | |
|---|---|
| Analytical Column ID | S71 |
| Material | X-Terra ™ MS C₁₈, 5 μm |
| Supplier | Waters, Eschborn, Germany |
| Dimensions | 150 mm × 2.1 mm (length, internal diameter) |

### 4.3 Mobile Phase

Aqueous phase: A: H₂O+0.1%
Organic phase: C: ACN
Gradient conditions:

| Time | Flow | % A | % C |
|---|---|---|---|
| 0.00 | 0.5 | 90 | 10 |
| 27.0 | 0.5 | 10 | 90 |
| 27.1 | 0.5 | 90 | 10 |
| 30.0 | 0.5 | 90 | 10 |

This example indicates that the salts of the disclosure, particularly the metal salts, e.g. alkali metal salts, are more stable than the acids, notably TRI 50c.

### Example 35

### In-Vitro Assay as Thrombin Inhibitor of Magnesium Salt of TRI 50C

### Thrombin Amidolytic Assay

TRI 50c magnesium salt (TRI 1405) was tested in a thrombin amidolytic assay.

### Reagents:

### Assay Buffer:

100 mM Na phosphate
200 mM NaCl (11.688 g/l)
0.5% PEG 6000 (5 g/l)
0.02% Na azide
pH 7.5
Chromogenic substrate S2238 dissolved to 4 mM (25 mg+10 ml) in water. Diluted to 50 uM with assay buffer for use in assay at 5 μM. (S2238 is H-D-Phe-Pip-Arg-pNA).

Thrombin obtained from HTI, via Cambridge Bioscience, and aliquoted at 1 mg/ml with assay buffer. Dilute to 10 ng/ml with assay buffer and then a further 1 in 3 for use in the assay.

### Assay:

110 μl assay buffer
50 ul 5 μg/ml thrombin
20 μl vehicle or compound solution

### 5 min at 37° C.

### 20 μl 50 μM S2238

Read at 405 nm at 37° C. for 10 minutes and record Vmax

### Results and Discussion:

In this assay the magnesium salt of TRI 50c shows the same activity as TRI 50b as an external control.

### Example 36

### Intravenous Administration of TRI 50C Sodium Salt

The pharmacokinetics (PK) and pharmacodynamics (PD) of TRI 50c sodium salt were studied in beagle dogs following intravenous administration.

The PD was measured as thrombin time and APTT using an automated coagulometer. Plasma concentrations were measured using an LCMS/MS method.

TRI 50c monosodium salt (108.8 g) was dissolved in 0.9% sodium chloride (100 ml) and dosed i.v. at 1.0 mg/kg (1.0 ml/kg over 30 seconds). Blood samples were taken into 3.8% tri-sodium citrate (1+8) at pre dose, 2, 5, 10, 20, 30, minutes post dose and then at 1, 2, 3, 4, 6, 8, 12 and 24 hours post dose. Plasma was prepared by centrifugation and frozen at minus 20° C. pending analysis.

### Results

The sodium salt was tolerated well with no adverse events for the total duration of the study. Male and female dogs responded similarly with a pharmacodynamic C max: at 2 minutes (thrombin time of 154 seconds raised from a base line of 14.3 seconds). Thrombin time was 26 seconds at one hour post dose.

There was an exceptionally good therapeutic ratio between the APTT and thrombin clotting time in dogs receiving the sodium salt at a dose of 1.0 mg/kg i.v. Thrombin clotting time was elevated 10.8 times above base line (154.4 seconds from 14.3 seconds) two minutes following dosing, compared to only 1.3 times elevation in the APTT (19 seconds to 25 seconds post dose).

### Example 37

### Residual n-Heptane of TRI 50C Calcium Salt

Salt prepared following the methods of Examples 1 and 3 was tested by headspace gas chromatography. Data are shown below:

Residual solvents: Headspace gas chromatography

| | |
|---|---|
| GC Parameter: | |
| Column: | DB-wax, 30 m, 0.32 mm ID, 5μ |
| Carrier Gas: | Helium 5.0, 80 kPas |
| Detector: | FID, 220° C. |
| Injector Temp: | 150° C. |
| Operating Conditions: | 35° C./7 min; 10° C./min up to 80° C./2 min; |
| | 40° C. up to 180° C./2 min |
| Injection volume: | 1 ml |
| Split: | On |
| Headspace Parameter: | |
| Oven temperature: | 70° C. |
| Needle temperature: | 90° C. |
| Transfer temperature: | 100° C. |
| Other parameters: | temper time: 15 min, GC-cycle time: 28 min, |
| | injection time: 0.03 min, duration: 0.4 min |

Calibration Standards: sample weight/dilution

| | | | concentration | area (average, |
|---|---|---|---|---|
| standard | weight (mg) | volume (ml) | (mg/ml) | n = 3) |
| n-heptane | 103.12 | 100 | 1.0312 | 2757.74756 |

| | | | concentration |
|---|---|---|---|
| sample no. | weight (mg) | volume (ml) | (mg/ml) |
| 1 | 100.84 | 5 | 20.17 |
| 2 | 99.12 | 5 | 19.82 |
| 3 | 100.03 | 5 | 20.01 |

n-heptane

| sample | concentration (mg/ml) | content (%) |
|---|---|---|
| 1 | 0.0010 | 0.0048 |
| 2 | 0.0009 | 0.0044 |
| 3 | 0.0010 | 0.0050 |
| | 0.00095 | 0.005 |

### Example 38

### HPLC Chromatograms

TRI 50c monosodium salt made by the method of Examples 1, 2 & 3 and TRI 50c hemicalcium salt made by the method of Examples 1, 2 & 4 were analysed by HPLC chromatography.

### 1. Method

### 1.1 Equipment and Software

| | |
|---|---|
| Autosampler | Waters Alliance 2795 |
| Pump | Waters Alliance 2795 |
| Column oven | Waters Alliance 2795 |
| Detection | Waters 2996 diode array, MS-ZQ single quad |
| Software version | Waters Millennium 4.0 |

### 1.2 Stationary Phase

| | |
|---|---|
| Analytical Column ID | S-71 |
| Material | XTerra ™ MS C₁₈, 5 μm |
| Supplier | Waters, Eschborn, Germany |
| Dimension | 150 mm × 2.1 mm (length, ID) |
| Pre-column ID | no pre-column |

Xterra MS C₁₈, 5 μm is a column packing material supplied by Waters Corporation, 34 Maple Street, Milford, Mass. 01757, US and local offices, as in years 2002/2003. It comprises hybrid organic/inorganic particles, consisting of spherical particles of 5 μm size, 125 Å pore size and 15.5% carbon load.

### 1.3 Mobile Phase

| | |
|---|---|
| Aqueous phase: | A: H₂O + 0.1% HCOOH |
| Organic phase: | C: ACN |

H₂O = H₂O by Ultra Clear water purification system
ACN = gradient grade acetonitrile

### Gradient Conditions

| time | | | flow | gradient |
|---|---|---|---|---|
| [min] | A % | C % | [mL/min] | shape |
| 0.0 | 90.0 | 10.0 | 0.5 | |
| 27.00 | 10.0 | 90.0 | 0.5 | linear |
| 27.10 | 90.0 | 10.0 | 0.5 | linear |
| 30.00 | 90.0 | 10.0 | 0.5 | linear |

### 1.4 Instrumental Parameters

| | |
|---|---|
| Flow | 0.5 mL-min⁻¹ |
| Temperature | 40 ± 5° C. |
| HPLC control | Waters Millennium Release 4.0 |
| Calculation | Waters Millenium 4.0 |

### 2, Parameters

### 2.1 Wavelength/Retention Time/Response Factors

TABLE
retention and detection parameter (k′ F: 0.5 ml/min, t0 = 0.9 mL/min)

| | | | | response | Reciprocal |
|---|---|---|---|---|---|
| | RetTime | λ | | factor | Response |
| Substance | [min] | [nm] | m/z | [area/μg] | factor |
| TRI 50c | 11.68 | 258 | 508.33 | 660 | 1 |
| Benzyl alcohol | 3.862 | 258 | n.d. | 1960 | 0.337 |
| Benzaldehyde | 6.13 | 258 | n.d. | 79939 | 0.0083 |
| Benzoic acid | 5.52 | 258 | n.d. | 5967 | 0.111 |
| Impurity I | 11.18 | 258 | 396.17 | 886 | 0.745 |
| Impurity II | 13.39 | 258 | 482.22 | 552 | 1.196 |

### 2.2 Linearity

Linearity Range 4000-10 μg/mL (Detection UV 258 nm)

TABLE
Linearity data UV 258 nm

| calibration | area | target conc. | conc. found¹ |
|---|---|---|---|
| solution | [μAU's] | [μg/mL] | [μg/mL] |
| Tri 50c | 5353 | 10 | 20.44 |
| Tri 50c | 5301 | 10 | 20.37 |
| Tri 50c | 65809 | 100 | 113.35 |
| Tri 50c | 66365 | 100 | 114.17 |
| Tri 50c | 172019 | 250 | 270.43 |
| Tri 50c | 162587 | 250 | 256.48 |
| Tri 50c | 339503 | 500 | 518.13 |
| Tri 50c | 326912 | 500 | 499.51 |
| Tri 50c | 659257 | 1000 | 991.02 |
| Tri 50c | 647495 | 1000 | 973.63 |
| Tri 50c | 1322371 | 2000 | 1971.72 |
| Tri 50c | 1305196 | 2000 | 1946.32 |
| Tri 50c | 2724410 | 4000 | 4045.24 |

¹recalculated with linear equation

### Linear Equation Parameters:

Y=6.75e+002X−8.45e+003

r=0.99975

r²=0.99950

Linearity Range 10-0.10 μg/mL (Detection SIR m/z 508,33)

TABLE
Linearity data SIR 508.33

| calibration | mean area | target conc. | conc. found¹ |
|---|---|---|---|
| solution | [μAU's] | [μg/mL] | [μg/mL] |
| Tri 50c | 2188860 | 0.01 | 0.022 |
| Tri 50c | 2702839 | 0.01 | 0.045 |
| Tri 50c | 3817226 | 0.1 | 0.094 |
| Tri 5Oc | 3833799 | 0.1 | 0.095 |
| Tri 50c | 23153550 | 1 | 0.947 |
| Tri 50c | 24646892 | 1 | 1.013 |
| Tri 50c | 223007852 | 10 | 9.765 |
| Tri 50c | 233753043 | 10 | 10.239 |

¹recalculated with linear equation

### Equation Parameter

Y=2.27e+007X+1.69e+006

r=0.99958

r²=0.99916

### 2.3 Quantitation Limit

The quantitation limit was determined using the signal to noise ratio criterion S/N>19,

UV 258 nm: 10 μg/mL

M/z 508.3: 0.1 μg/mL

### 2.4 Precision

| | | | | Retention |
|---|---|---|---|---|
| | Target concentration | | Amount | time |
| Injection | [μg/mL] | Area | [μg/mL] | [min] |
| 1 | 250 | 165805 | 261.24 | 11.690 |
| 2 | 250 | 168644 | 265.44 | 11.662 |
| 3 | 250 | 167858 | 264.27 | 11.686 |
| 4 | 250 | 166947 | 262.93 | 11.692 |
| 5 | 250 | 166925 | 262.89 | 11.679 |
| 6 | 250 | 166294 | 261.96 | 11.696 |
| Mean | | 167079 | 263.12 | 11.684 |
| Std. Dev. | | 1033 | 1.528 | 0.01 |
| % RSD | | 0.6 | 0.6 | 0.1 |

### 2.5 Robustness

TABLE
robustness data; Standard 250 μg/mL aqueous solution
(containing < 1% ACN)

| calibration | temp./time area | | recovery |
|---|---|---|---|
| solution | [° C./h] | area [μAU's] | [%] |
| 250 μg/mL Tri50c | — | 172020 | — |
| 250 μg/mL Tri50c | 4° C. 16 h | 166294 | 96.67 |
| 2.5 μg/mL TRI50c | — | 88034891 | — |
| 2.5 μg/mL TRI50c | 37° C. 4 h | 88833175 | 100.9 |

### REFERENCES

1. ICH HARMONISED TRIPARTITE GUIDELINE. TEXT ON VALIDATION OF ANALYTICAL PROCEDURES Recommended for Adoption at Step 4 of the ICH Process on 27 Oct. 1994 by the ICH Steering Committee
2. FDA Reviewer Guidance. Validation of chromatographic methods. Center for Drug Evaluation and Research. November 1994
3. USP 23. <621> Chromatography
4. L. Huber. Validation of analytical Methods. LC-GC International February 1998
5. Handbuch Validierung in der Analytik. Dr. Stavros Kromidas (Ed.) Wiley-VCH Verlag. 2000. ISBN 3-527-29811-8

### 3. Results

### 3.1 Sample Name: TRI 50c Monosodium Salt

Injection volume: 10 μL

| | Ret Time | | Area | Peak Height |
|---|---|---|---|---|
| Name | (Min) | Area % | [μAU's] | μAU |
| TRI 50c | 12.136 | 100.0000 | 604.27228 | 32.05369 |

3.2 Sample Name: TRI 50c hemicalcium salt

Injection volume: 10 μL

| | Ret Time | | Area | Peak Height |
|---|---|---|---|---|
| Name | (Min) | Area % | [μAU's] | μAU |
| TRI 50c | 12.126 | 100.0000 | 597.11279 | 32.29640 |

The disclosed methods have been used to obtain salts substantially free of C—B bond degradation products, in particular salts containing no such products in an amount detectable by HPLC, specifically the method of Example 38. The disclosed methods have been used to obtain salts substantially free of Impurity I, in particular containing no Impurity I in an amount detectable by HPLC, specifically by the method of Example 38. The disclosed methods have been used to obtain salts substantially free of Impurity IV, in particular containing no Impurity IV in an amount detectable by HPLC, specifically by the method of Example 38.

### Example 39

### Determination of Diastereomeric Excess

TRI 50b, crude, contains three chiral centres. Two of them are fixed by the use of enantiomerically pure amino acids ((R)-Phe and (S)-Pro). The third one is formed during the synthesis. The favoured epimer is the desired TRI 50b, Isomer I (R,S,R-TRI 50b). Both epimers of TRI 50b are clearly baseline separated by the HPLC method, thus allowing determination of the diasteromeric excess (de) of TRI 50b.

TRI 50d is not stable under the conditions applied for HPLC purity determination, but decomposes rapidly on sample preparation to TRI 50c, so that TRI 50d and TRI 50c show the same HPLC traces.

The two isomers of TRI 50c are not baseline separated in HPLC, but both isomers are clearly visible. This becomes obvious, when TRI 50b, crude (mixture of both isomers) is converted with phenylboronic acid to TRI 50c, crude. Both isomers of TRI 50c are observed in HPLC nearly at the same relation as before in TRI 50b, crude.

Upon synthesis of TRI 50d from TRI 50b, crude, only one diastereoisomer is precipitated. In this case HPLC shows only one peak for TRI 50c, where a very small fronting is observed. Precipitation from dichloromethane/diethylether removes the fronting efficiently. The level of removal of Isomer II cannot be quantified by this HPLC method. Therefore samples before reprecipitation and after one and two reprecipitations were esterified with pinacol and the resulting samples of TRI 50b analysed by HPLC. Thus a de of 95.4% was determined for the crude sample. The reprecipitated sample resulted in a de of 99.0% and finally the sample that was reprecipitated twice showed a de of 99.5%.

These results clearly show the preferred precipitation of Isomer I, whereas Isomer II remains in solution.

It will be appreciated from the foregoing that the disclosure provides boronic acid salts useful for pharmaceutical purposes and which feature one or more of the following attributes: (1) improved hydrolytic stability; (2) improved stability against deboronation; and (3), in any event, not suggested by the prior art.

The selection of active ingredient for a pharmaceutical composition is a complex task, which requires consideration not only of biological properties (including bioavailability) but also of physicochemical properties desirable for processing, formulation and storage. Bioavailability itself is dependent on various factors, often including in vivo stability, salvation properties and absorption properties, each in turn potentially dependent on multiple physical, chemical and/or biological behaviours.

### Example 40

### Short Duration Gastrically Accessible Formulation

A formulation of TRI 50c hemicalcium salt (TGN 167) was produced in the form of round, white tablets 11 mm in diameter. Each tablet contained 75 mg of TRI 50c incorporated as the calcium salt (TGN 167) with the following components and composition:

| Name of Ingredient | Unit Formula | Standard | Function |
|---|---|---|---|
| Tablet Core | | | |
| Active Substance | | | |
| TRI 50c-04 (calcium salt) | 75 mg as TRI 50c free | Trigen | Active ingredient |
| | acid | specification | |
| Excipients | | | |
| Sodium Starch Glycolate Type A | 17 mg | PhEur | Disintegrant |
| Hydroxypropylcellulose (Klucel EF) | 17 mg | PhEur | Binder |
| Magnesium Stearate | 4.25 mg | PhEur | Lubricant |
| Microcrystalline Cellulose | to 425 mg | PhEur | Diluent |
| (Avicel PH102) | | | |
| Purified Water | * | PhEur | Granulating |
| | | | agent |
| Tablet Coat | | | |
| Sub coat | | | |
| Hypromellose | 13 mg (8.5-17 mg) | PhEur | Film agent |
| Purified Water | ** | PhEur | Solvent |

The choice of excipient and composition was made with the primary goal of achieving rapid dissolution.

### Trial Protocol

TGN 167 tablets described above were administered via the oral route to 10 healthy male subjects as single doses (randomised double blind placebo controlled study). The group consisted of 10 healthy male volunteers, 9 of whom received active compound and one of whom received placebo. An amendment was introduced for the last panel, allowing 15 subjects (instead of 9) to receive a 600 mg dose. The interval between each dose of TGN 167 was one week minimum. The allocation of the placebo was such that each volunteer would only receive placebo once during the course of the study.

Orally administered doses were: 75 mg (one tablet), 150 mg (two tablets), 300 mg (four tablets) and 600 mg (eight tablets).

### Study Results

1) No clinically significant findings were detected in any safety assessments. There were no adverse clinical events of either a general or cardiovascular nature during the study period of 24 hours for any dose of TGN 167.

2) Oral administration of TGN 167 induced a dose-related increase in the Thrombin Time (TT), reaching a peak within 2 to 3 h after administration, with maximum mean values being approximately 2.5 to 7 fold higher than at pre-dose. A fall in TT to 1.5 fold baseline values occurred within 4 hours after administration. All TT had returned to baseline 10 hours after administration.

The above results indicate that TRI 50c salts have suitable pharmacodynamic properties of a short duration oral antithrombotic drug, namely a rapid rise in thrombin time after administration followed shortly thereafter by a fall in TT to a level only slightly above baseline at which clinically significant anticoagulation is not expected.

### Example 41

### TGN255 Sachet Formulations to be Dissolved in 150 mL Water

TGN 255 is the monosodium salt of TRI 50c. In the following tables, the amount of TGN 255 is expressed in terms of the equivalent amount of TRI 50c.

Powders

| Ingredient | g/sachet | Function |
|---|---|---|
| TGN255 | 0.6 g | Active Ingredient |
| Sucrose | 10 g | Sweetener and diluent |
| Acesulfame K | 0.04 g | Sweetener |
| Citric acid | 0.2 g | Flavour enhancer |
| Lemon Flavour | qs | Flavour |
| Total wt: | ~10 g | |
| TGN255 | 0.6 g | Active Ingredient |
| Sorbitol powder | 5 g | Sweetener and diluent |
| Aspartame | 0.04 g | Sweetener |
| Aniseed Flavour | qs | Flavour |
| Total wt: | ~5.7 g | |

Granules

| Ingredient | g/sachet | Function |
|---|---|---|
| TGN255 | 0.6 g | Active Ingredient |
| Flavour | qs | Flavour |
| Sorbitol | 8.9 g | Sweetener and diluent |
| Lutrol F68 (Poloxamer 188) | 0.5 g | Binder |
| Water | * | Binder |
| Total wt: | ~10 g | |

* Use 50% w/w water to granulate. Not included in final formulation.

| | | |
|---|---|---|
| TGN255 | 0.6 g | Active Ingredient |
| Flavour | qs | Flavour |
| Citric acid | 0.2 g | Flavour enhancer and buffer |
| Sodium citrate | 0.3 g | Flavour enhancer and buffer |
| Sorbitol | 8.8 g | Sweetener and diluent |
| Lutrol F68 (Poloxamer 188) | 0.2 g | Binder |
| Ethanol 96% | ND* | |
| Total wt: | ~10 g | |

*Use 20% w/w ethanol to granulate. Not included in final formulation.

The powders and granules are packaged in sachets.

### Example 42

### Comparative Stability

The stability of TRI 50c sodium salt and TRI 50c sodium salt calcium salt have been studied in studies similar design and conditions. In both studies the active pharmaceutical ingredient was stored in gray sealed double bags within a PE/PP screw cap closed cylinder. The packaging allows moisture transfer and the study was designed to allow the investigation into the effects of moisture and oxygen on the stability of these TRI 50c salts.

The results observed after 12 months storage are summarised in the tables below.

Results
Sodium salt, data in % w/w

| | | | T = 1, | T = 1, | | T = 3, | T = 3, | T = 12, |
|---|---|---|---|---|---|---|---|---|
| | | T = 1, | 25° C./ | 40° C./ | T = 3, | 25° C./ | 40° C./ | 25° C./ |
| | T = 0 | −20° C. | 60% r.h. | 75% r.h. | −20° C. | 60% r.h. | 75% r.h | 60% r.h. |
| Appearance | Powdery | powdery | powdery | powdery | powdery | powdery | viscous | |
| Color | White | white | white | white | white | white | brown | |
| Tri50c (w/w %) | 101.1 | 103.1 | 99.5 | 90.3 | 102.5 | 95.3 | 48.6 | 70.5 |
| Tri50c (w/w %, | 101.5 | 104.3 | 103.6 | 94.5 | — | 100.4 | 52.2 | 71.2 |
| LOD, corr.) | | | | | | | | |

Results
Calcium salt, data in % w/w

| | | | T = 1, | T = 1, | | T = 3, | T = 3 | T = 12 |
|---|---|---|---|---|---|---|---|---|
| | | T = 1, | 25° C./ | 40° C./ | T = 3, | 25° C./ | 40° C./ | 25° C./ |
| | T = 0 | −20° C. | 60% r.h. | 75% r.h. | −20° C. | 60% r.h. | 75% r.h. | 60% r.h. |
| Appearance | powdery, | powdery | powdery | powdery | powdery | powdery | powdery | |
| | odourless | | | | | | | |
| Color | white | white | white | white | white | white | white | |
| TRI 50c (% peak area) | 99.4 | 99.1 | 98.3 | 95.2 | 99.2 | 97.5 | 71.2 | 96.9 |
| Tri50c (w/w %, | (99.7) | (102.7) ^{x} | (101.5) | (100.6) | (103.0) ^{x} | (104.3) | (82.0) | (94.8 w/w) |
| LOD corrected) | | | | | | | | |

LOD = loss on drying

### Discussion

The data in this example indicate that calcium salts of boronic acids are more stable than the corresponding sodium salts. It is contemplated that the same benefit may be provided by zinc.

The present disclosure includes the subject matter of the following paragraphs (which are not consecutively numbered):

1. A solid phase pharmaceutical formulation adapted for reconstitution into the liquid phase before it enters the stomach and comprising a boronic acid of formula (I), or a salt or prodrug thereof:

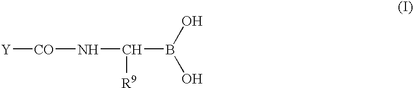

wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen (F, Cl, Br or I).

2. A formulation of paragraph 1 wherein R⁹ is an alkoxyalkyl group.

3. A formulation of paragraph 1 or paragraph 2 wherein YCO— comprises an amino acid which binds to the S2 subsite of thrombin, the amino acid being N-terminally linked to a moiety which binds the S3 subsite of thrombin.

4. A formulation of paragraph 1 or paragraph 2 wherein Y is an optionally N-terminally protected dipeptide which binds to the 53 and 52 binding sites of thrombin and the peptide linkages in the acid are optionally and independently N-substituted by a C₁-C₁₃ hydrocarbyl optionally containing in-chain or in-ring nitrogen, oxygen or sulfur and optionally substituted by a substituent selected from halo, hydroxy and trifluoromethyl.

5. A formulation of paragraph 4 wherein said dipeptide is N-terminally protected and all the peptide linkages in the acid are unsubstituted.

6. A formulation of paragraph 4 or paragraph 5 wherein the S3-binding amino acid residue is of R configuration, the S2-binding residue is of S configuration, and the fragment —NHCH(R⁹)—B(OH) is of R configuration.

7. A formulation of any of paragraphs 1 to 6 wherein the boronic acid has a Ki for thrombin of about 100 nM or less.

8. A formulation of paragraph 7 wherein the boronic acid has a Ki for thrombin of about 20 nM or less.

9. A reconstitutable oral formulation of a boronic acid of formula (II) or a salt, prodrug or prodrug salt thereof:

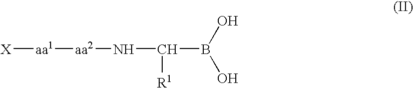

where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members;
R¹ is a group of the formula —(CH₂)ₛ-Z, where s is 2, 3 or 4 and Z is —OH, —OMe, —OEt or halogen (F, Cl, Br or I).

10. A formulation of paragraph 9 wherein aa¹ is selected from Phe, Dpa and wholly or partially hydrogenated analogues thereof.

11. A formulation of paragraph 9 wherein aa¹ is selected from Dpa, Phe, Dcha and Cha.

12. A formulation of any of paragraphs 9 to 11 wherein aa¹ is of R-configuration.

13. A formulation of paragraph 9 wherein aa¹ is (R)-Phe (that is, D-Phe) or (R)-Dpa (that is, D-Dpa).

14. A formulation of paragraph 9 wherein aa¹ is (R)-Phe.

15. A formulation of any of paragraphs 9 to 14 wherein aa² is a residue of an imino acid of formula (IV)

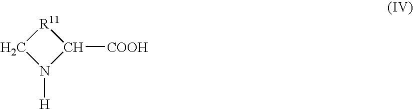

where R¹¹ is —CH₂—, —CH₂—CH₂, —CH₂═CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups.

16. A formulation of paragraph 15 wherein aa² is of S-configuration.

17. A formulation of paragraph 15 wherein aa² is an (S)-proline residue.

18. A formulation of paragraph 9, wherein aa¹-aa² is (R)-Phe-(S)-Pro (that is, D-Phe-L-Pro).

19. A formulation of any of paragraphs 9 to 18 wherein R¹ is 2-bromoethyl, 2-chloroethyl, 2-methoxyethyl, 3-bromopropyl, 3-chloropropyl or 3-methoxypropyl.

20. A formulation of any of paragraphs 9 to 18 wherein R¹ is 3-methoxypropyl.

21. A formulation of any of paragraphs 9 to 20 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by one or more (e.g. 1, 2, 3, 4 or 5) halogens (e.g. F), for example at least at the 4-position, and/or by 1, 2 or 3 substituents selected from amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.

22. A formulation of paragraph 21 wherein said 5 to 13-membered cyclic group is aromatic or heteroaromatic.

23. A formulation of paragraph 22 wherein said 5 to 13-membered cyclic group is phenyl or a 6-membered heteroaromatic group.

24. A formulation of any of paragraphs 9 to 20 wherein X is R⁶—(CH₂)ₚ—C(O)— or R⁶—(CH₂)ₚ—O—C(O)— and p is 0, 1 or 2.

25. A formulation of any of paragraphs 9 to 20 wherein X is benzyloxycarbonyl.

26. A formulation of paragraph 9 wherein the boronic acid is of formula (VIII):

X—(R)-Phe-(S)-Pro-(R)-Mpg-B(OH)₂ (VIII).

27. A formulation of any of paragraphs 1 to 26 wherein the boronic acid is in the form of a pharmaceutically acceptable salt thereof, e.g. with monovalent or divalent counter-ions.

28 A formulation of paragraphs 27 which comprises a salt of the peptide boronic acid with an alkali metal or a strongly basic organic nitrogen-containing compound.

29. A formulation of paragraph 28 wherein the strongly basic organic nitrogen-containing compound is a guanidine, a guanidine analogue or an amine.

30. A formulation of paragraph 27 wherein the salt is a salt of the boronic acid with a metal.

31. A formulation of paragraph 26 which comprises a salt of the boronic acid with an alkali metal, an aminosugar, a guanidine or an amine of formula (XI):

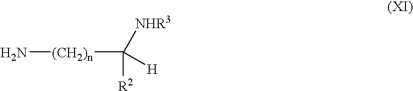

where n is from 1 to 6, R² is H, carboxylate or derivatised carboxylate, R² is H, C₁-C₄ alkyl or a residue of a natural or unnatural amino acid.

32. A formulation of paragraph 26 which comprises a salt of the boronic acid with a guanidine or with an amine of formula (XI):

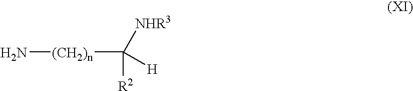

where n is from 1 to 6, R² is H, carboxylate or derivatised carboxylate, R³ is H, C₁-C₄ alkyl or a residue of a natural or unnatural amino acid.

33. A formulation of paragraph 32 which comprises a guanidine salt of the boronic acid.

34. A formulation of paragraph 33 which comprises a salt of the boronic acid with L-arginine or an L-arginine analogue.

35. A formulation of paragraph 34 wherein the L-arginine analogue is D-arginine, or the D- or L-isomers of homoarginine, agmatine [(4-aminobutyl) guanidine], NG-nitro-L-arginine methyl ester, or a 2-amino pyrimidines.

36. A formulation of paragraph 33 which comprises a salt of the boronic acid with a guanidine of formula (VII)

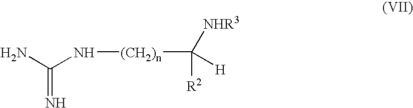

where n is from 1 to 6, R² is H, carboxylate or derivatised carboxylate, R³ is H, C₁-C₄ alkyl or a residue of a natural or unnatural amino acid.

37. A formulation of paragraph 36, wherein n is 2, 3 or 4.

38. A formulation of paragraph 36 or paragraph 37 where the derivatised carboxylate forms a C₁-C₄ alkyl ester or amide.

39. A formulation of any of paragraphs 36 to 38 wherein the compound of formula (VII) is of L-configuration.

40. A formulation of paragraph 33 which comprises an L-arginine salt of the peptide boronic acid.

41. A formulation of paragraph 32 which comprises a salt of the boronic acid with an amine of formula (IX).

42. A formulation of paragraph 41, wherein n is 2, 3 or 4.

43. A formulation of paragraph 41 or paragraph 42 where the derivatised carboxylate forms a C₁-C₄ alkyl ester or amide.

44. A formulation of any of paragraphs 41 to 43 wherein the amine of formula (IX) is of L-configuration.

45. A formulation of paragraph 41 which comprises an L-lysine salt of the boronic acid.

46. A formulation of any of paragraphs 1 to 26 which comprises an alkali metal salt of the boronic acid.

47. A formulation of paragraph 46 wherein the alkali metal is potassium.

48. A formulation of paragraph 46 wherein the alkali metal is sodium.

49. A formulation of paragraph 46 wherein the alkali metal is lithium.

50. A formulation of paragraph 27 which comprises an aminosugar salt of the boronic acid.

51. A formulation of paragraph 50 wherein the aminosugar is a ring-opened sugar.

52. A formulation of paragraph 51 wherein the aminosugar is a glucamine.

53. A formulation of paragraph 50 wherein the aminosugar is a cyclic aminosugar.

54. A formulation of any of paragraphs 50 to 53 wherein the aminosugar is N-unsubstituted.

55. A formulation of any of paragraphs 50 to 53 wherein the aminosugar is N-substituted by one or two substituents.

56. A formulation of paragraph 55 wherein the or each substituent is a hydrocarbyl group.

57. A formulation of paragraph 55 wherein the or each substituent is selected from the group consisting of alkyl and aryl moieties.

58. A formulation of paragraph 57 wherein the or each substituent is selected from the group consisting of C₁, C₂, C₃, C4, C₅, C6, C₇ and C₈ alkyl groups

59. A formulation of any of paragraphs 55 to 58 wherein there is a single N-substituent.

60. A formulation of paragraph 50 wherein the glucamine is N-methyl-D-glucamine.

61. A formulation of any of paragraphs 27 to 60 which comprises boronate ions derived from the peptide boronic acid and has a stoichiometry consistent with the boronate ions carrying a single negative charge.

62. A formulation of any of paragraphs 27 to 60 wherein the salt consists essentially of acid salt (that is, wherein one B—OH group remains protonated).

63. A formulation of any of paragraphs 27 to 62 wherein the salt comprises a boronate ion derived from the peptide boronic acid and a counter-ion and wherein the salt consists essentially of a salt having a single type of counter-ion.

82. The use of a salt of any of paragraphs 1 to 63 for the manufacture of a parenteral medicament for a treatment recited in any of paragraphs 76 to 81.

83. A reconstitutable oral pharmaceutical formulation comprising a combination of (i) an acid, prodrug or salt of any of paragraphs 1 to 63 and (ii) a further pharmaceutically active agent.

84. A reconstitutable oral pharmaceutical formulation comprising a combination of (i) an acid, prodrug or salt of any of paragraphs 1 to 63 and (ii) another cardiovascular treatment agent.

85. A formulation of paragraph 84 wherein the other cardiovascular treatment agent comprises a lipid-lowering drug, a fibrate, niacin, a statin, a CETP inhibitor, a bile acid sequestrant, an anti-oxidant, a IIb/IIIa antagonist, an aldosterone inhibitor, an A2 antagonist, an A3 agonist, a beta-blocker, acetylsalicylic acid, a loop diuretic, an ace inhibitor, an antithrombotic agent with a different mechanism of action, an antiplatelet agent, a thromboxane receptor and/or synthetase inhibitor, a fibrinogen receptor antagonist, a prostacyclin mimetic, a phosphodiesterase inhibitor, an ADP-receptor (P₂ T) antagonist, a thrombolytic, a cardioprotectant or a COX-2 inhibitor.

86. The use of an acid, prodrug or salt of any of paragraphs 1 to 63 for the manufacture of a reconstitutable oral medicament for treating, for example preventing, a cardiovascular disorder in co-administration with another cardiovascular treatment agent.

87. A reconstitutable oral medicament comprising a salt of a boronic acid which is a selective thrombin inhibitor and has a neutral aminoboronic acid residue capable of binding to the thrombin S1 subsite linked through a peptide linkage to a hydrophobic moiety capable of binding to the thrombin S2 and S3 subsites, the salt comprising a cation having a valency n and having an observed stoichiometry consistent with a notional stoichiometry (boronic acid:cation) of n:1.

88. A medicament of paragraph 87 wherein the boronic acid has a Ki for thrombin of about 100 nM or less.

89. A medicament of paragraph 87 wherein the boronic acid has a Ki for thrombin of about 20 nM or less.

90. A dosage form comprising a sodium salt of Cbz-(R)-Phe-(S)-Pro-(R)-Mpg-B(OH)₂, the dosage form being a sachet containing a powder or granules containing said salt or an effervescent tablet.

91. A pharmaceutical product comprising a sealed container containing in the form of a finely divided solid or granules, ready for reconstitution to form a liquid oral formulation, a therapeutically effective amount of a boronic acid of formula II or a salt or prodrug thereof, the boronate salt consisting essentially of a single pharmaceutically acceptable base addition salt of a boronic acid formula (II):

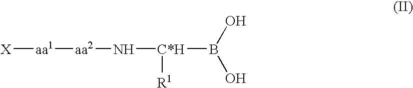

where:
X is H (to form NH₂) or an amino-protecting group; aa¹ is an amino acid of R-configuration having a hydrocarbyl side chain containing no more than 20 carbon atoms and comprising at least one cyclic group having up to 13 carbon atoms;
aa² is an imino acid of S-configuration having from 4 to 6 ring members;
C* is a chiral centre of R-configuration; and
R¹ is a group of the formula —(CH₂)ₛ-Z, where s is 2, 3 or 4 and Z is —OH, —OMe, —OEt or halogen (F, Cl, Br or I).

92. The product of paragraph 91 wherein:

X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group;
aa¹ is selected from D-Phe, D-Dpa, D-Cha and Dcha;
aa² is Pro; and
R¹ is 2-ethoxyethyl or 3-methoxypropyl.

93. A pharmaceutical formulation adapted for oral administration after combining with a liquid, and comprising
a) a first species selected from (a) boronic acids of formula (I), (b) boronate anions thereof, and (c) any equilibrium form of the aforegoing (e.g. an anhydride):

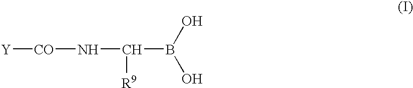

wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen (F, Cl, Br or I); and
(b) a second species selected from the group consisting of pharmaceutically acceptable metal ions, said metal ions having a valency of n, lysine, arginine and aminosugars,
   wherein the formulation has an observed stoichiometry of first to second species essentially consistent with a notional stoichiometry of 1:1 except where the second species is a metal ion having a valency of greater than 1, in which case the observed stoichiometry is essentially consistent with a notional stoichiometry of n:1.

94. The formulation of paragraph 93 which has the characteristic that, after the formulation if not in an aqueous carrier is placed in one, it has a Ki for thrombin of about 20 nM or less.

95. The formulation of paragraph 93 or 94 in which R⁹ is 3-methoxypropyl and the second species is sodium ions, potassium ions, lithium ions or lysine.

96. The formulation of any of paragraphs 93 to 95 which is in the form of fine particles for combining with a liquid to form a liquid formulation.

97. A product comprising, in a form for reconstitution into a drinking solution, a compound selected from boronic acids of formula II and their salts, e.g. a salt consisting essentially of a mono-alkali metal salt of an acid of the formula Cbz-(R)-Phe-(S)-Pro-(R)-Mpg-B(OH)₂, the compound optionally being in admixture with an organic acid.

98. The product of paragraph 97 in which the salt is in unit dosage form.

99. The product of paragraph 97 wherein the unit dosage form contains from about 0.2 mol to 1.5 mol of the salt.

100. The product of any of paragraphs 97 to 99 which includes no isotonicity agent.

101. The product of any of paragraphs 97 to 100 which includes an anti-microbial preservative and a flavour agent.

102. A product comprising, in the form of an effervescent tablet, a salt consisting essentially of a mono-alkali metal salt of an acid of the formula Cbz-(R)-Phe-(S)-Pro-(R)-Mpg-B(OH)₂, the salt containing no more than small amounts of other epimers of said acid, the product optionally further containing an organic acid.

103. The product of paragraph 102 which contains from about 0.2 mol to 1.5 mol of the salt.

It will be apparent from the aforegoing that the oral anticoagulant activity provided by the disclosed boronic acids, as represented by TRI 50c administered as salt forms is well suited to haemodialysis. The rapid onset of anticoagulant activity, its magnitude and the offset of anticoagulation provide the desired anticoagulant acitivity, its magnitude and the offset of anticoagulation provide the desired anticoagulation profile to prevent coagulation in the haemodialysis circuit for the typical duration of a haemodialysis session. A drinking solution reconstituted from a solid preparation will provide a satisfactory dosing regimen.

1. An oral dosage form of a compound selected from boronic acids which have a neutral thrombin PI domain linked to a hydrophobic moiety capable of binding to the thrombin S2 and S3 subsites, and salts, prodrugs and prodrug salts of such acids, the dosage form comprising a solid phase formulation comprising the compound and being adapted for reconstitution of the formulation to form a liquid preparation.

2. The dosage form of claim 1 wherein the thrombin P1 domain comprises a neutral aminoboronic acid residue.

3. The dosage form of claim 1 wherein the boronic acid is of formula (I): 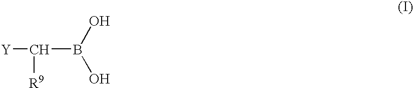
wherein
Y comprises a moiety which, together with the fragment —CH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is from 2, 3, 4 or 5 and W is —OH or halogen, wherein halogen is F, Cl, Br or I.

4. The dosage form of claim 3 wherein R⁹ is an alkoxyalkyl group.

5. The dosage form of claim 3 wherein Y comprises
an amino group bonded to structural fragment —CH(R⁹)—B(OH)₂, and
a hydrophobic moiety which is linked to said amino group and which, together with said structural fragment, has affinity for the substrate binding site of thrombin.

6. The dosage form of claim 5 wherein Y comprises an amino acid which binds to the S2 subsite of thrombin, the amino acid being N-terminally linked to a moiety which binds the S3 subsite of thrombin.

7. The dosage form of claim 6 wherein Y is an optionally N-terminally protected dipeptide which binds to the S3 and S2 binding sites of thrombin and the peptide linkages in the acid are optionally and independently N-substituted by a C₁-C₁₃ hydrocarbyl optionally containing in-chain or in-ring nitrogen, oxygen or sulfur and optionally substituted by a substituent selected from halo, hydroxy and trifluoromethyl.

8. The dosage form of claim 7 wherein the S3-binding amino acid residue is of (R) configuration, the S2-binding residue is of (S) configuration, and the fragment —NHCH(R⁹)—B(OH)₂ is of (R) configuration.

9. The dosage form of claim 1 wherein said compound is a pharmaceutically acceptable base addition salt of a said acid.

10. An oral pharmaceutical dosage form adapted to be reconstituted either
prior to administration into a liquid for oral administration, or
in the mouth,
and comprising a compound selected from boronic acids of formula (III) and salts, prodrugs and prodrug salts thereof:
where: 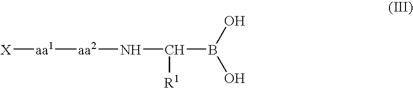
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members; and
R¹ is a group of the formula —CH₂)ₛ-Z, where s is 2, 3 or 4 and Z is —OH, —OMe, —OEt or halogen, wherein halogen is F, Cl, Br or I.

11. The dosage form of claim 10 wherein aa¹ is selected from Phe, Dpa and wholly or partially hydrogenated analogues thereof.

12. The dosage form of claim 10 wherein aa² is a residue of an imino acid of formula (IV) 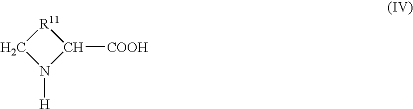
where R¹¹ is —CH₂—, —CH₂—CH₂—, —CH₂═CH₂—, —S—CH₂—, —S—C(CH₃)₂— or —CH₂—CH₂—CH₂—, which residue, when the ring contained therein is 5- or 6-membered, is optionally substituted at one or more —CH₂— groups by from 1 to 3 C₁-C₃ alkyl groups.

13. The dosage form of claim 10 wherein aa¹ is of (R)-configuration, aa² is of (S)-configuration and the fragment —NH—CH(R¹)—B(OH)₂ is of (R)-configuration.

14. The dosage form of claim 10 wherein R¹ is 2-bromoethyl, 2-chloroethyl, 2-methoxyethyl, 3-bromopropyl, 3-chloropropyl or 3-methoxypropyl.

15. The dosage form of claim 10 where X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 and R⁶ is H or a 5 to 13-membered cyclic group optionally substituted by one or more halogens and/or by 1, 2 or 3 substituents selected from amino, nitro, hydroxy, a C₅-C₆ cyclic group, C₁-C₄ alkyl and C₁-C₄ alkyl containing, and/or linked to the cyclic group through, an in-chain O, the aforesaid alkyl groups optionally being substituted by a substituent selected from halogen, amino, nitro, hydroxy and a C₅-C₆ cyclic group.

16. The dosage form of claim 10 wherein the boronic acid is of formula (VIII):
X—(R)-Phe-(S)-Pro-(R)-Mpg-B(OH)₂ (VIII).

17. The dosage form of claim 9 wherein the salt comprises a salt of the boronic acid with a metal.

18. The dosage form of claim 17 wherein the metal comprises an alkali metal.

19. The dosage form of claim 1 which comprises boronate ions derived from the boronic acid and has a stoichiometry consistent with the boronate ions carrying a single negative charge.

20. The dosage form of claim 1 which comprises:
a pharmaceutical formulation which contains said compound and is in the form of powder or granules; and
a sealed container in which the formulation is contained and from which the formulation is to be dispensed for reconstitution.

21-22. (canceled)

23. The dosage form of claim 20 wherein the container is a sachet.

24. The dosage form of claim 1 wherein the solid phase formulation is a pharmaceutical formulation in the form of an effervescent tablet which contains an effervescent system, or is a fast melt pharmaceutical formulation.

25. (canceled)

26. The dosage form of claim 20 which comprises from about 0.2 to about 1.5 mol of the compound, calculated on the basis of the boronic acid.

27. (canceled)

28. The dosage form of claim 1 which is adapted to be reconstituted to form a solution having a volume of from about 50 ml to about 150 ml.

29. A pharmaceutical formulation comprising a pharmaceutically acceptable base addition salt of the acid Cbz-(R)-Phe-(S)-Pro-(R)-Mpg-B(OH)₂, the formulation being in the form of a powder or granules in a sachet or of an effervescent tablet.

30. A method of making an oral dosage form for preventing thrombosis, comprising:
reacting a boronic acid which has a neutral thrombin P1 domain linked to a hydrophobic moiety capable of binding to the thrombin S2 and S3 subsites with a base selected from the group consisting of basic metal compounds and organic nitrogen-containing compounds having a pKb of at least 7, to form a reaction product; and
formulating the reaction product into a solid phase formulation which comprises the reaction product and is adapted for reconstitution of the formulation to form a liquid preparation.

31. A method for the manufacture of a medicament to be reconstituted to form a drinkable preparation, comprising making the medicament with a compound as defined in claim 1.

32-33. (canceled)

34. A method of preparing an anticoagulant preparation, comprising reconstituting, into a liquid preparation for oral administration a solid phase formulation comprising:
a) a first species selected from the group consisting of a boronic acid of formula (I) below, said acid when in the form of a boronate anion thereof, an equilibrium form of said boronic acid and of said boronate ion, and combinations thereof: 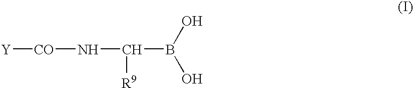
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen, wherein halogen is F, Cl, Br or I; and
(b) a second species selected from the group consisting of pharmaceutically acceptable metal ions and strongly basic organic nitrogen-containing compounds.

35. A method of inhibiting thrombin in the treatment of disease, comprising administering perorally to a subject in need thereof a therapeutically effective amount of a compound as defined in claim 1, said compound being put into solution or suspension from a solid phase formulation prior to the compound entering the stomach.

36. The method of claim 35, wherein the compound is put into solution or suspension by reconstituting with a liquid prior to administration or in saliva in the mouth.

37. A method of preventing thrombosis in the haemodialysis circuit of a patient, comprising reconstituting into a drinkable preparation a solid formulation comprising a salt as defined in claim 9, and orally administering the drinkable preparation.

38-39. (canceled)

40. A method of preventing deep vein thrombosis during an airplane flight in a subject comprising administering to the subject a therapeutically effective amount of a compound as defined in claim 1.

41-42. (canceled)

43. A method of preventing thrombosis in intermittent apheresis comprising administering a therapeutically effective amount of a compound as defined in claim 1, wherein said intermittent apheresis is not hemodialysis.

44. The method of claim 43, wherein the intermittent apheresis is extracorporeal liver detoxification.

45. The method of claim 43, wherein the compound is an oral medicament or is a parenteral medicament.

46. A method for the prevention of thrombosis in the haemodialysis circuit of a patient undergoing haemodialysis, comprising administering a therapeutically effective amount of a compound selected from boronic acids which have a neutral thrombin P1 domain linked to a hydrophobic moiety capable of binding to the thrombin S2 and S3 subsites, and salts, prodrugs and prodrug salts of such acids, the compound not being a base addition salt of such a boronic acid.

47. The method of claim 46 wherein the boronic acid is of formula (I): 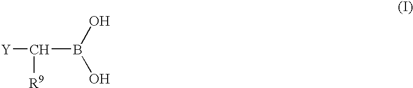
wherein
Y comprises a moiety which, together with the fragment —CH(R⁹)—B(OH)₂, has affinity for the substrate binding site of thrombin and R⁹ is an alkoxyalkyl group, and wherein Y comprises an amino acid which binds to the S2 subsite of thrombin, the amino acid being N-terminally linked to a moiety which binds the S3 subsite of thrombin, the S3-binding amino acid residue is of (R) configuration, the S2-binding residue is of (S) configuration, and the fragment —CH(R⁹)—B(OH)₂ is of (R) configuration.

48. The method of claim 46 wherein the boronic acid is of formula (III): 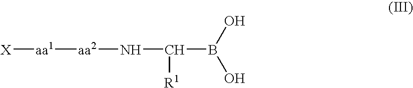
where:
X is H (to form NH₂) or an amino-protecting group;
aa¹ is an amino acid having a hydrocarbyl side chain containing no more than 20 carbon atoms and comprising at least one cyclic group having up to 13 carbon atoms;
aa² is an imino acid having from 4 to 6 ring members;
R¹ is a group of the formula —(CH₂)ₛ-Z, where s is 2, 3 or 4 and Z is —OH, —OMe, —OEt or halogen, wherein halogen is F, Cl, Br or I.

49. The method of claim 46 wherein the boronic acid is a compound designated TRI 50c of the following formula
Cbz-(R)-Phe-(S)-Pro-(R)-Mpg-B(OH)₂.

50. The dosage form of claim 10 wherein the prodrugs are boronic acid derivatives capable of hydrolysing to release the free boronic acid.

51. A method for preventing flight deep vein thrombosis or thrombosis in intermittent apheresis, wherein said intermittent apheresis is not hemodialysis, comprising administering a therapeutically effective amount of a composition of matter comprising
a) a first species selected from the group consisting of a boronic acid of formula (I) below, said acid when in the form of a boronate anion thereof, an equilibrium form of said boronic acid and of said boronate ion, and combinations thereof: 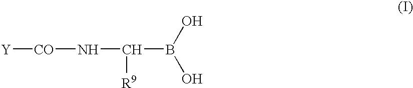
wherein
Y comprises a hydrophobic moiety which, together with the aminoboronic acid residue —NHCH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen, wherein halogen is F, Cl, Br or I; and
(b) a second species selected from the group consisting of pharmaceutically acceptable metal ions and strongly basic organic nitrogen-containing compounds.

52. An aqueous solution comprising a pharmaceutically acceptable base addition salt of a boronic acid which has a neutral thrombin P1 domain linked to a hydrophobic moiety capable of binding to the thrombin S2 and S3 subsites, the solution having a pH of about 9 or more.

53. The solution of claim 52 wherein the pH is about 9 to about 9.5.

54. An aqueous solution comprising a pharmaceutically acceptable base addition salt of a boronic acid which has a neutral thrombin P1 domain linked to a hydrophobic moiety capable of binding to the thrombin S2 and S3 subsites and a pharmaceutically acceptable organic acid, the solution having a pH of from about 4 to about 8.
